(19) United States
(12) Patent Application Publication
(10) Pub. No.: US 2012/0028811 A1
(43) Pub. Date: Feb. 2, 2012

(54) DEVICE FOR RAPID IDENTIFICATION OF NUCLEIC ACIDS FOR BINDING TO SPECIFIC CHEMICAL TARGETS

(75) Inventors: Harold G. Craighead, Ithaca, NY (US); John T. Lis, Ithaca, NY (US); Seungmin Park, Berkeley, CA (US); So Youn Kim, Seoul (KR); Jiyoung Ahn, Seoul (KR); Minjoung Jo, Seoul (KR)

(73) Assignees: DONGGUK UNIVERSITY, Seoul (KR); CORNELL UNIVERSITY, Ithaca, NY (US)

(21) Appl. No.: 13/059,223

(22) PCT Filed: Aug. 17, 2009
(86) PCT No.: PCT/US09/54097

§ 371 Date: Oct. 10, 2011

Related U.S. Application Data
(62) Division of application No. 61/089,291, filed on Aug. 15, 2008.

(51) Int. Cl. C40B 10/00; C07H 21/00; B32B 37/14; G03F 7/20; B32B 37/02; C40B 60/08; C40B 40/06

(52) U.S. Cl. 506/1; 506/37; 536/23.1; 506/16; 430/296; 156/60

(57) ABSTRACT

The present invention relates to microfluidic chips and their use in SELEX. The microfluidic chip preferably includes a reaction chamber that contains a high surface area material that contains target. One preferred high surface area material is a sol-gel derived material. Methods of making the microfluidic chips are described herein, as are uses of these devices to select aptamers against the target.

This application claims the benefit of U.S. Provisional Patent Application Ser. No. 61/089,291 filed Aug. 15, 2008, which is hereby incorporated by reference in its entirety.

This invention was made with government support under grant numbers ECS-9731293 and ECS-9876771 by the National Science Foundation. The government has certain rights in this invention.

### FIELD OF THE INVENTION

The present invention is directed to a device and method for rapid identification of nucleic acids that bind specifically to biological and chemical targets.

### BACKGROUND OF THE INVENTION

The process known as SELEX (Systematic Evolution of Ligands by Exponential Enrichment) is an evolutionary, in vitro combinatorial chemistry process used to identify aptamers binding to a ligand or target from large pools of diverse oligonucleotides. SELEX is an excellent system for isolating aptamers from a random pool under specific customizable binding conditions. The SELEX process has provided an alternative for generating single stranded DNA or RNA oligonucleotides that bind tightly and specifically to given ligands or targets. (Tuerk et al., Science 249:505-510 (1990); Ellington A., Curr Biol 4:427-429 (1994); Ellington et al., Nature 346:818-822 (1990)). SELEX experiments have been exploited to investigate the functional and structural aspects of nucleic acids, and the identified aptamers have become an important tool for the research of molecular diagnostics, molecular recognition, molecular biology, and molecular evolution (Uphoff et al., Curr Opin Struct Biol 6:281-288 (1996)).

In SELEX, aptamer selection is enriched by the repetition of successive steps of target binding and removal of unbound oligonucleotides, followed by elution, amplification, and purification of the selected oligonucleotides. SELEX involves repetitive rounds of two processes: (i) partitioning or selection of high affinity aptamers from low affinity aptamers by an affinity method and (ii) amplification of selected aptamers by the polymerase chain reaction (PCR). Aptamers are typically selected from large pools or libraries (≧10¹⁵ individuals) of random DNA or RNA sequences by the affinity selection method in the partitioning step of the SELEX process. The single stranded DNA or RNA, so called “aptamer,” are artificial specific oligonucleotides with the ability to bind to non-nucleic acid target molecules with high affinity and specificity (Jenison et al., Science 263:1425 (1994); Patel et al., J Mol Biol 272:645-664 (1997); Clark et al., Electrophoresis 23:1335-1340 (2002)) Due to their unique properties, aptamers promise to revolutionize many areas of natural and life sciences ranging from affinity separation to diagnostics and treatment of diseases such as cancers and viral infections (Tang et al., Anal Chem 79:4900-4907 (2007); Gopinath, S., Archives of Virology 152:2137-57 (2007)).

Aptamers have several advantages over antibodies. They are smaller, more stable, can be chemically synthesized, and can be fluorescently labeled for their detection without affecting their affinity. In contrast to antibody development, their development for toxic targets (when used for antibody generation) or targets with low or no immunogenicity is feasible (Mann et al., Biochem Biophy Res Comm 338:1928-1934 (2005)). Moreover, due to their easy and rapid preparation and versatility, they have become advantageous tools for the validation of intra- and extracellular targets. (Gopinath, S., Anal Bioanal Chem. 387:171-182 (2007)). A set of aptamers could also provide ways of selectively perturbing a subset of connections of a “hub” protein. (Shi et al., Proc Nat'l Acad Sci USA 104:3742-3746 (2007)).

Microfluidics refers to systems that handle very small volumes of liquid (˜10⁻⁹-10⁻¹⁸ liters) using micrometer sized channels. Handling of small volumes offers high speed chemical reactions by decreasing diffusion time and provide accurate control over sample liquids acquired during delivery, exchange and positioning of chemicals to the required position. With microfabrication techniques, microfluidics also realizes integration of fluidic elements such as micropump, microvalve, microheater, etc. in a single chip so that it makes it possible to automate chemical processes on the chip. For these reasons, microfluidics can be broadly utilized in the field of chemistry, biology, medicine and engineering to analyze samples with high speed and high throughput (Whitesides, G., Nature 442:368-373 (2006)).

Traditional SELEX systems in practice are repetitive, time-consuming, and unsuitable for high-throughput selections. While the SELEX process itself has been well-established, the relatively low throughput prohibits studies that require a large number of distinct aptamers, such as for proteomics studies for biomarker identity. One way to increase the speed of aptamer generation and selection power by SELEX is through automation and miniaturization of the process. Recently, progress has been made toward the miniaturization of macro-scale techniques for the development of rapid and high-throughput analysis. Benefits from miniaturization include 1) small sample consumption, 2) ability of high-throughput analysis, 3) self-containment, 4) decrease in cross contamination, and 5) integration of multiple functions (Gopinath, S, Anal Bioanal Chem. 387:171-182 (2007)). The SELEX process used to isolate specific RNA aptamers can be automated, significantly reducing the time required for isolation and amplification of oligonucleotides sequences capable of high affinity binding to specific target molecules of interest. Recently, several microfluidic protocols have been introduced to develop a faster SELEX process, significantly reducing the time required for aptamer generation by SELEX from months/weeks to a few days (Hybarger, et al., Anal Bioanal Chem 384:191-198 (2006); Windbichler, et al., Nat. Protoc. 1:637-640 (2006); Eulberg, et al., Nucleic Acids Research 33:e45 (2005)). Most advances in developing the SELEX process, have aimed at improving the efficiency of selection (Bunka et al., Nat Rev Micro 4:588-596 (2006)). However, these studies have not employed miniaturized or multiplexed aptamer selection.

The SELEX process could potentially be standardized, giving significant advantages in terms of fast analysis, reduced cost and high-throughput analysis if the system is integrated into a chip-based, microfluidic environment. Chip-based enzymatic assays (Hadd et al., Anal Chem 69:3407-3412 (1997); Joseph W., Electrophoresis 23:713-718 (2002)) and immunoassays (Wang et al., Anal Chem 73:5323-5327 (2001); Sato et al., Anal Chem 73:1213-1218 (2001)) have documented such advantages.

There are several also disadvantages to conventional SELEX selection methods. One problem with the conventional selection process is that the aptamer is selected to have affinity for a target molecule that is bound to a stationary support rather than one that is free in solution. The evolutionary process of SELEX, rather than converging on an aptamer that has affinity for the desired target, selects an aptamer that binds a molecule similar to the target (i.e., the membrane bound derivative thereof). It has been shown that aptamers selected to bind cAMP actually had stronger affinity for cAMP analogs modified at the C8 position, the same position where the target was tethered to the stationary. support (Koizumi et al., Biochem. 39:8983-8992 (2000)). Thus, the effect of the stationary support is amplified when selecting aptamers for smaller ligands, because smaller ligands only have a limited number of functionalities that can interact with the aptamer and attaching the ligand to a stationary support further reduces the availability of these functionalities.

Other problems are introduced by the stationary support itself. It has been suggested that the rinsing step used in conventional SELEX, where the active sequences are removed from the column with a solution of free target may bias against aptamers with very high affinity for the target (Klug et al., Mol. Biol. Rep., 1994; 20:97-107 (1994)). A major concern is kinetic bias where it is almost impossible to elute very strongly interacting sequences from a chromatography column. Sequences with high affinity for the target would not wash off the column easily. This can also appear when the aptamer is highly specific for bound (immobilized) target while the elution is done with free, unbound target. Therefore, it may be impossible to recover sequences with picomolar or lower dissociation constants from the selection column.

The present invention is directed to overcoming these and other deficiencies in the art.

### SUMMARY OF THE INVENTION

A first aspect of the present invention is directed to a microfluidic device that includes a substrate having one or more fluid channels extending between an inlet and an outlet, a molecular binding region within the one or more fluid channels, wherein the molecular binding region includes a target molecule, and a heating element adjacent to the molecular binding region. Preferably, the molecular binding region includes a high surface area material that includes the target molecule. Kits containing these devices are also disclosed herein.

A second aspect of the present invention is directed to a method of selecting a nucleic acid aptamer for binding to one or more target molecules. The method includes providing a microfluidic device according to the first aspect of the invention and introducing a population of nucleic acid molecules into the microfluidic device under conditions effective to allow the nucleic acid molecules to bind specifically to the target molecule. The method further includes removing from the microfluidic device substantially all nucleic acid molecules that do not bind specifically to the target molecule, heating the heating element to cause denaturation of nucleic acid molecules that bind specifically to the target molecule, and recovering nucleic acid molecules that bind specifically to the target molecule. The recovered nucleic acid molecules are aptamers that have been selected for their binding to the target molecule.

A third aspect of the present invention is directed to a method of selecting a nucleic acid aptamer for binding to one or more target molecules. This method includes providing a microfluidic device that includes a substrate with one or more fluid channels extending between an inlet and an outlet, and one or more molecular binding regions within the one or more fluid channels, wherein the one or more molecular binding regions each contain a target molecule. The method further includes introducing a population of nucleic acid molecules into the microfluidic device under conditions effective to allow nucleic acid molecules to bind specifically to the target molecule(s), removing from the microfluidic device substantially all nucleic acid molecules that do not bind specifically to the target molecule(s), denaturing the nucleic acid molecules that bind specifically to the target molecule(s), and recovering nucleic acid molecules that bind specifically to the target molecule(s). The recovered nucleic acid molecules are aptamers that have been selected for their binding to the target molecule.

A fourth aspect of the present invention relates to one or more aptamers identified in Tables 1-8 (except for SEQ ID NOS: 24, 70, and 81).

A fifth aspect of the present invention relates to a method of making a microfluidic SELEX device of the invention. The method includes applying a sol-gel material including a target molecule onto a surface of a first body component, and allowing solvent evaporation to occur, thereby forming a porous matrix that includes the target molecule; and then sealing a second body component onto the first body component, whereby the first and second body components together define a microfluidic device having an inlet, an outlet, and at least one microfluidic channel between the inlet and outlet, whereby the porous matrix is in fluid communication with the at least one microfluidic channel.

The microfluidic SELEX chip described herein offers a number of significant advantages that substantially improve the outcome of SELEX. One significant advantage of a preferred embodiment is that nanoporous sol-gel material, which is utilized to immobilize target protein(s) in one or more microfluidic chambers of the microfluidic device, supports the competitive binding of an aptamer library to the target proteins. A localized heat source is used selectively to elute the specific high affinity aptamers that bind the target protein. The ability to immobilize protein in sol-gel material makes it an excellent candidate for the miniaturized devices since sol-gel does not require affinity capture tags or recombinant proteins, and therefore allows for entrapment of various proteins in their native state without any linking agents (Gill I., Chemistry of Materials 13:3404-3421 (2001), which is hereby incorporated by reference in its entirety). This overcomes the limitation of conventional SELEX where aptamers are selected against bound targets. This reduces the possibility of kinetic traps where a strongly binding aptamer sequence is never eluted from the target. Because the partitioning or separation of the non-binding aptamers from the binding aptamers is a critical and often rate limiting step in the SELEX processes, the microfluidic system of the present invention is a quicker and more efficient alternative.

The present invention also allows for high-throughput and optionally multiplexed selection, and characterization of aptamers specific for targets. The microfluidic device can be used in serial assays or parallel assays, increasing the throughput together with decreasing the assay time, sample volume, and cost. Experimental procedures for the optimized separation of the aptamers have also been disclosed.

The Examples presented herein demonstrate, using a sol-gel based microfluidic SELEX system of the present invention, i.e., SELEX-on-a-chip, the selection of a number of aptamers for TATA binding protein (“TBP,” Yokomori et al., Genes & Dev. 8:2313-2323 (1994), which is hereby incorporated by reference in its entirety). These results demonstrate that TBP aptamers can be efficiently isolated using the SELEX-on-a chip, confirming the utility of the device for supporting a high throughput SELEX method. The microfluidic SELEX systems of the present invention greatly improved the selection efficiency by reducing the number of selection cycles used to produce high affinity aptamers by as much as 50 percent. As confirmation of its efficiency and effectiveness, use of the microfluidic SELEX system produced high affinity TBP aptamers that were identical or homologous to those isolated previously by conventional filter-binding SELEX.

Finally, the microfluidic SELEX systems of the present invention can be used for screening aptamers against multiple distinct target molecules, using a single chip in combination with automated SELEX machinery. This should greatly enhance the capacity for identifying novel aptamer molecules that are selective against one or more targets of interest.

### BRIEF DESCRIPTION OF THE DRAWINGS

FIG. 1A is a plain view of a SELEX microfluidic chip, and FIG. 1B is an enlarged image illustrating the relative position of a sol-gel deposited on an electrode of the chip. The diameter of the illustrated sol-gel is about 300 μm. FIG. 1C is a schematic diagram of the SELEX microfluidic chip (exploded) along with the accompanying system for carrying out the delivery of fluids to the SELEX microfluidic chip. The direction of the flow through the microchip is from the negative sol-gel (N) to spot 4. The order of aptamer collection is in the reverse direction (from 4 to 3 to 2, 1, and then N) of the flow, which prevents unwanted heating from buffer passing over the other electrodes.

FIG. 2 is schematic illustrating a fabrication process for a SELEX microfluidic chip.

FIGS. 3A-B show the microfluidic SELEX process and the microfluidic chip. FIG. 3A illustrates the aptamer screening process using the sol-gel derived microfluidic chip. Briefly, random RNA aptamer pool, reagents and buffers were delivered through capillaries to the chip. Aptamers with specific binding affinity can be entrapped by the target protein in the sol-gel droplets located in the chambers of the microfluidic device (also described as molecular binding regions). Five sets of sol-gel droplets were spotted evenly along the microfluidic channels (N is the negative control; 1 has entrapped yeast TATA Binding Protein (TBP); 2 has yeast Transcription Factor IIA (TFIIA); 3 has yeast Transcription Factor IIB (TFIIB); 4 has human Heat Shock Factor 1 (HSF1)). The distance between the droplets was kept at 1 cm to prevent possible unwanted heating from the other heating electrodes. Bound aptamers against each target were eluted sequentially by heating the individual aluminum microheaters. FIG. 3B illustrates the microfabricated sol-gel chip. This embodiment includes a glass slide with a set of aluminum electrodes and a PDMS lid, with the lid and slide together defining a microfluidic channel having five distinct chambers. The microfluidic parts embossed on the PDMS lid include 170 μm deep and 300 μm wide microchannels and five hexagonal chambers with a side length of 1 mm. The typical volume of a single microdroplet of sol-gel is around 7 nl and each droplet can hold 30 fmoles protein inside the nanoporous structure. For incubation and reaction purposes, five hexagonal chambers were designed in this device. The volume of this hexagonal chamber and the connecting channel between the chambers are 0.22 μl and 0.41 μμl, respectively. The finished dimension of the microfluidic chip is 75 mm×25 mm×5 mm.

FIG. 4 shows the Scanning Electron Microscope (SEM) image of a sol-gel. Two different types of pores were observed. The diameters of the big pore group are between about 100 to about 200 nm. The small pore group is between about 20 to about 30 nm in diameter. These pores are spread evenly over the surface of the sol-gel. The scale bar shown in the image is 1 μm.

FIGS. 5A-D show the fluorescence intensity of sol-gel spots on the aluminum electrodes. SYBR-Green I labeled dsDNA (100 bp, 1 nM) in sol-gel spot was denatured by individual electrode heating. The fluorescence intensity vs. time with various powers on electrodes is plotted along with the exponential decay model (red line). Each graph is accompanied by a series of fluorescence micrographs of sol-gel spots at 20 seconds intervals. The 1^{e} points were calculated from the fluorescence intensity from each graph to obtain the appropriate time and power. FIG. 5A shows 100 mW, 39.5 sec; FIG. 5B shows 424 mW, 7.4 sec; FIG. 5C shows 536 mW, 3.3 sec; and FIG. 5D shows 645 mW, 1.8 sec.

FIGS. 6A-B show the binding of TATA DNA to TBP graphically. FIG. 6A is an intensity vs. time graph. For the binding of TATA DNA to the sol-gels with embedded TBP, the intensity vs. time graph can be fit to the exponential decay model. A power of 450 mW was delivered to the electrode. The acquired half-life time of the intensity decrease was 6.4 sec. The intensity reduction is believed to be due to the aptamer release from the immobilized target protein. FIG. 6B shows bright field micrographs of the sol-gel after binding with Cy-3 labeled TATA DNA and subsequent elution.

FIG. 7A-D show gel electrophoresis band images of the collected RNA. To visualize the RNA in the gel electrophoresis, the RNA was reverse transcribed using its primers and amplified by PCR. Four samples with different RNA concentration were prepared (FIG. 7A shows 2.6 pmole, FIG. 7B shows 13 pmole, FIG. 7C shows 77 pmole, and FIG. 7D shows 130 pmole). The order of the band in the images is M (marker-ladder DNA), N (Negative control), 1, 2, 3 and 4. Negative bands show almost no or low signal compared with the others. The marker indicates that the expressed band in the gel is the right size. This means aptamers bound specifically to the target, e.g., protein in the sol-gel, rather than non-specifically to the sol-gel itself.

FIGS. 8A-B show band intensity comparison between collected samples with different RNA concentrations. FIG. 8A shows the electropherogram of the standard marker (Lane M) and collected aptamers (Lane N, 50, 30, 5). Lane N is from the negative control sol-gel. The initial amount of the aptamers are 3.56 μg (indicated as 50 in the graph), 2.14 μg (30), and 356 ng (5). Band intensities were calculated using a Matlab program. The intensity is proportional to the amount of the aptamers in selection. The band intensity from the negative control is almost same as background. FIG. 8B illustrates the band intensity graphically.

FIG. 9 shows the results for electrophoretic mobility shift assay (EMSA) of the collected RNAs from the multiplexed sol-gel chip. The affinity of the collected aptamers to their target proteins was tested. All collected RNAs were labeled with P³², a radio isotope tag. These RNAs were then incubated with 0 nM, 50 nM of target proteins (TBP and TFIIB). EMSA tests indicate that RNA aptamers show specific affinity only to the target protein: #12 to TBP only and #4 to TFIIB only, and not vice versa.

FIGS. 10A-C show improved in vitro selection cycle efficiency. FIG. 10A shows three new products (G5′, G6′ and G7′) of RNA pool were obtained from the conventional SELEX round 4 (G4), 5 (G5) and 6 (G6) by using the microfluidic SELEX chip. The conventional SELEX for TFIIB started with a starting pool of 2×10¹) sequences. FIG. 10B shows the electrophoretic mobility shift assay (EMSA) with P³² labeled RNA pool (G6′ and G7′) from the microfluidic SELEX chip. This was performed with increasing concentrations of TFIIB (0, 2.5, 12.5, 62.5 nM). FIG. 10C shows EMSA results in which aptamer (G7′) does not bind to TBP or TFIIA, but binds with high affinity to TFIIB. All proteins used had a concentration of 200 nM.

FIG. 11 comparatively illustrates the process used for microfluidic SELEX versus conventional SELEX process. Several TBP aptamers have been isolated after the 11^{th} round of conventional SELEX, which uses filter binding. The microfluidic SELEX method of the present invention required fewer cycles of SELEX than the conventional SELEX method. The microfluidic SELEX was performed after two rounds of conventional SELEX on a filter. Filter binding products were converted to RNA and injected into a microfluidic device. The focus of this study was on TBP (TATA Binding Protein) microfluidic SELEX. TBP aptamers (ms 3, ms 4, ms 5, and ms 6) were sequenced after every cycle of SELEX, and their sequences are listed in Tables 1-4 infra. The experiments confirmed that the microfluidic SELEX device of the present invention can hold and enrich the specific aptamers against the target protein, which in this Example was TBP. Upon comparison to the conventional SELEX aptamers, the aptamers obtained from microfluidic SELEX were classified into two groups (matched and newly selected aptamers).

FIGS. 12A-B show the aptamer binding assay using a sol-gel array chip. FIG. 12A shows the assay design, with each well having sol-gel spots containing TBP printed onto a PMMA coated 96 well chip along with positive (P) and negative (N) controls as illustrated. The RNA aptamer pool for the ms-6 round was end labeled with Cy-3. FIG. 12B shows the individual binding activity of newly selected aptamers. The binding activity was calculated by using the fluorescent intensity of sol-gel spot. As a negative control, a binding assay was performed without aptamer and the signal intensity was measured on TBP droplet positions. ms-6.4, ms-6.16 and ms-6.38 belong to group I (matched aptamer marked with star); all other aptamers were new.

FIGS. 13A-B show the fluorescent assay and the binding affinity of aptamers to TBP. Individual binding affinity of aptamers (ms-6.12, ms-6.15, ms-6.16, ms-6.18, ms-6.24, and ms-6.26) to TBP were measured by sol-gel chip assay. In one well, 5 types of duplicate sol-gel microdroplets with different protein concentrations (from 0 to 400 nM) were spotted. The average volume of one droplet was around 50 nl. FIG. 13A shows the microdroplet positions for the distribution of different concentrations of TBP, and the fluorescent intensity observed at these spots. Six TBP aptamers were added into each well and the resulting signals appeared after the assay. As shown in FIG. 13B, the binding affinities (K_{d}) were measured by the mean value of spot intensities. All assays were performed in duplicate. K_{d} values for the aptamers are: ms-6.12≈2.7 nM; ms-6.15≈13.2 nM; ms-6.16≈8.3 nM; ms-6.18≈4.5 nM; ms-6.24≈92.53 nM; and ms-6.26≈10.56 nM.

FIGS. 14A-F show the Mfold-generated secondary structures for the aptamer sequences. Lowest free energy of aptamer structures are entered in parenthesis. FIG. 14A shows aptamer ms-6.12 (ΔG=−18.5) (SEQ ID NO: 68), FIG. 14B shows ms-6.15 (ΔG=−13.9) (SEQ ID NO: 69), FIG. 14C shows ms-6.16 (ΔG=−33.7) (SEQ ID NO: 70), FIG. 14D shows ms-6.18 (ΔG=−28.23) (SEQ ID NO: 72), FIG. 14E shows ms-6.24 (ΔG=−20.80) (SEQ ID NO: 74), and FIG. 14F shows ms-6.26 (ΔG=−20.60) (SEQ ID NO: 75). Each aptamer is composed of 99 nucleotides (nt) with central 50-nucleotide variable region (shown in uppercase letters) flanked by 49-nucleotides of the constant primer binding region (shown in lowercase letters) on both 5′ end and 3′ end (SEQ ID NO: 82).

FIG. 15 is a schematic illustration of a 96-chamber multiplex microfluidic SELEX chip that includes a PDMS pump-valve system having a pneumatic valve controller and two pumps.

### DETAILED DESCRIPTION OF THE INVENTION

One aspect of the present invention relates to a microfluidic device that can be used for performing high-throughput screening of aptamer pools using a modified SELEX process. Preferred embodiments of the microfluidic device also overcome several deficiencies of conventional SELEX, afford improved efficiency in aptamer selection, and ensure selection of aptamers that bind to unmodified target molecules.

The microfluidic device includes a substrate which comprises one or more fluid channels extending between an inlet and an outlet, a molecular binding region within the one or more fluid channels, wherein the molecular binding region comprises a target molecule, and a heating element adjacent to the molecular binding region.

The microfluidic device includes an aggregation of separate parts, for example, but not limited to, fluid channels, capillaries, joints, chambers, layers, and heating elements, which when appropriately mated or joined together, form the microfluidic device of the invention. The microfluidic devices preferably, though not necessarily, include a top portion, a bottom portion, and an interior portion, one or more of which substantially define the channels and chambers of the device.

In one embodiment, the bottom portion is a solid substrate that is substantially planar in structure, and which has a substantially flat upper surface. A variety of substrate materials may be used to form the bottom portion. The substrate materials should be selected based upon their compatibility with known microfabrication techniques, for example, photolithography, wet chemical etching, laser ablation, air abrasion techniques, injection molding, embossing, and other techniques. The substrate materials are also generally selected for their compatibility with the full range of conditions to which the microfluidic devices may be exposed, including extremes of pH, temperature, salt concentration, and/or application of electric fields.

Preferred substrate materials include, without limitation, glass, pyrex, glass ceramic, polymer materials, semiconductor materials, and combinations thereof. In some preferred aspects, the substrate material may include materials normally associated with the semiconductor industry in which microfabrication techniques are regularly employed, including, e.g., silica based substrates such as glass, quartz, silicon or polysilicon, as well as other substrate materials, such as gallium arsenide and the like. In the case of semiconductive materials, it will often be desirable to provide an insulating coating or layer, e.g., silicon oxide or silicon nitride, over the substrate material, particularly where electric fields are to be applied.

Exemplary polymeric materials include, without limitation, plastics such as polymethylmethacrylate (PMMA), polycarbonate, polytetrafluoroethylene (TEFLON™), polyvinylchloride (PVC), polydimethylsiloxane (PDMS), and polysulfone. Other plastics can also be used. Such substrates are readily manufactured from microfabricated masters, using well known molding techniques, such as injection molding, embossing or stamping, or by polymerizing the polymeric precursor material within a mold. Such polymeric substrate materials are known for their ease of manufacture, low cost and disposability, as well as their general inertness to most extreme reaction conditions. These polymeric materials may include treated surfaces, for example, derivatized or coated surfaces, to enhance their utility in the microfluidic system, or for example to provide enhanced fluid direction.

Ideally, the material used to build the interior portion, which at least partially defines the microfluidic channels, should also be biocompatible and resistant to biofouling. Because the active surface area of the device is only a few μm², the material used to form the interior portion should have a resolution that enables the structuring of both small cross-sectional area channels (on the order of about 2-3 μm width and about 1-2 μm height) and larger cross-sectional area channels (on the order of about 25 to about 500 μm width and/or height, more preferably about 50 to about 300 μm). Several existing materials, widely used for the fabrication of fluidic channels, can address these basic needs.

Two categories can be distinguished among them: those based on glasses, such as glass, Pyrex, quartz, etc. (Ymeti et al., Biosens. Bioelectron. 20:1417-1421 (2005), which is hereby incorporated by reference in its entirety); and those based on polymers such as polyimide, photoresist, SU-8 negative photoresist, polydimethylsiloxane (“PDMS”), silicone elastomer PDMS (McDonald et al., Electrophoresis 21:27-40 (2000), which is hereby incorporated by reference in its entirety), liquid crystal polymer, Teflon, etc.

While the glass materials have great chemical and mechanical resiliency, their high cost and delicate processing make them less frequently used for this kind of application. In contrast, polymers have gained wide acceptance as the materials of choice for fluidics applications. Moreover, structuring technologies involved in their use, such as bonding, molding, embossing, melt processing, and imprinting technologies, are now well developed (Mijatovic et al., Lab on a Chip 5:492-500 (2005), which is hereby incorporated by reference in its entirety). An additional advantage of polymer-based microfluidic systems is that valves and pumps made with the same material are readily integrated (Unger et al., Science 288:113-116 (2000), which is hereby incorporated by reference in its entirety).

PDMS and SU-8 resist are particularly well studied as raw materials for the construction of microfluidic systems. While both of them are optically transparent, their mechanical and chemical comportment are strongly disparate. SU-8 is stiffer (Blanco et al., J Micromechanics Microengineering 16:1006-1016 (2006), which is hereby incorporated by reference in its entirety) than PDMS, and so the structuring techniques of these two materials are different. PDMS is also subject to wall collapse, depending on the aspect ratios of the channels (Delamarche et al., Adv. Materials 9:741-746 (1997), which is hereby incorporated by reference in its entirety). Their chemical properties are an important aspect for the desired application. They both have a hydrophobic surface after polymerization, which can lead to an attachment of the proteins onto the PDMS walls, and can fill the channel in case of small cross-section. Both the surface of PDMS and of SU-8 can be treated with a surfactant or by plasma to become hydrophilic (Nordstrom et al., J Micromechanics Microengineering 14:1614-1617 (2004), which is hereby incorporated by reference in its entirety). The composition of SU-8 can also be modified before its structuring to become hydrophilic after polymerization (Chen and Lee, J Micromechanics Microengineering 17:1978-1984 (2007), which is hereby incorporated by reference in its entirety). Fouling of the channel surface via nonspecific binding is an obvious concern for any microfluidic application. Anecdotal evidence suggests that SU-8 is less prone to this, but it is important to note that chemical treatment methods are also available for improving the performance of PDMS (Lee and Voros, Langmuir 21:11957-11962 (2004), which is hereby incorporated by reference in its entirety).

The substrate materials can also be a combination of a glass or Pyrex base and a polymer lid, which together define the one or more fluid channels. The channels and/or chambers of the microfluidic devices are typically fabricated as microscale grooves or indentations formed into the upper surface of the substrate or bottom surface of the polymer lid using the above described microfabrication techniques. The lower surface of the top portion of the microfluidic device, which top portion typically comprises a second planar substrate, is then overlaid upon and bonded to the surface of the bottom substrate, sealing the channels and/or chambers (the interior portion) of the device at the interface of these two components. Bonding of the top portion to the bottom portion may be carried out using a variety of known methods, depending upon the nature of the substrate material. For example, in the case of glass substrates, thermal bonding techniques may be used which employ elevated temperatures and pressure to bond the top portion of the device to the bottom portion. Polymeric substrates may be bonded using similar techniques, except that the temperatures used are generally lower to prevent excessive melting of the substrate material. Alternative methods may also be used to bond polymeric parts of the device together, including acoustic welding techniques, or the use of adhesives, for example, UV curable adhesives.

The heating element can be made of any materials which are good conductors of both heat and electricity. According to one preferred embodiment, the heat element is a metal that can withstand the exposure to harsh or continually changing chemical and fluid environments such as extremes of pH, temperature, salt concentration, and application of electric fields. The expansion and contraction properties of the material used to form the heating element should be compatible with the corresponding properties of the substrate materials, such that the expansion does not lead to dissociation from the substrate or other complications in the microfluidic device. Exemplary metals include, without limitation, aluminum, silver, gold, platinum, copper, and alloys.

In certain embodiments, the microfluidic device of the present invention can also include a thermally conductive coating that encapsulates the heating element such that fluid passing through the fluid channels does not directly contact the heating element. This can be done to prevent the exposure of the metal parts of the heating element from corroding when in contact with harsh chemical environments. Preferred coating materials include, without limitation, glass, pyrex, glass ceramic, and polymer materials. One preferred polymer coating for this purpose is a poly(meth)acrylate or urethane-acrylate coating material.

The microfluidic chips of the present invention are not limited in their physical dimensions and may have any dimensions that are convenient for a particular application. For the sake of compatibility with current laboratory apparatus, microfluidic chips with external sizes of a standard microscope slide or smaller can be easily made. Other microfluidic chips can be sized such that the chips fit a standard size used on an instrument, for example, the sample chamber of a mass spectrometer or the sample chamber of an incubator. The chambers within the microfluidic chips of the present invention may have any shape, such as rectangular, square, oval, circular, or polygonal. The chambers or channels in the microfluidic chips may have square or round bottoms, V-shaped bottoms, or U-shaped bottoms. The shape of the chamber bottoms need not be uniform on a particular chip, but may vary as required by the particular SELEX being carried out on the chip. The chambers in the microfluidic chips of the present invention may have any width-to-depth ratio. The chambers (wells) and channels in the microfluidic chips of the present invention may have any volume or diameter which is compatible with the requirements of the sample volume being used. The chambers (wells) or channels can function as a reservoir, a mixer, or a place where chemical or biological reactions take place.

The microfluidic device of the present invention preferably includes at least one chamber positioned between the inlet and outlet and in fluid communication with the one or more fluid channels. The molecular binding region is preferably contained within the at least one chamber.

In one embodiment, the microfluidic devices include two or more chambers per channel. Each of the two or more chambers may contain the same target molecule, or the two or more chambers can contain different target molecules. Thus, devices loaded with multiple targets can be used for parallel SELEX on multiple targets. This embodiment can overcome the limitation of performing SELEX on one target at a time by providing microfluidic chips with two or more densely packed chambers in which targets are embedded in the sol-gel materials and aptamer selection can be conducted in parallel. This allows for selection of the aptamers against multiple targets.

As demonstrated in the accompanying Examples, one format of this embodiment includes five chambers on a single channel, allowing for tetra-plex SELEX against four distinct molecular targets along with a single control chamber. Other multiplex formats are also contemplated including, without limitation, 24-plex, 96-plex, 120-plex, 240-plex, and higher. These higher SELEX multiplex schemes can be performed using a single fluid channel or multiple fluid channels. Where multiple fluid channels are provided, the different channels can be used, for example, in different rounds of selection using the microfluidic SELEX procedure of the present invention.

In a preferred embodiment of the invention, the molecular binding region includes a high surface area material that contains the target molecule. This high surface area molecule is used to contain or entrap the target molecule such that the target molecules can bind effectively to the nucleic acid aptamers while remaining in their native state. That is, the target molecules preferably are not chemically modified in any way that may affect the availability of binding sites on the surface thereof. The molecular binding region is preferably included in one or more chambers of the microfluidic chip. By high surface area material, it is intended that the material be sufficiently porous to allow for diffusion of the nucleic acid molecules into the pores of the material where the nucleic acid molecules can contact and, if possible, bind specifically to the target molecules contained therein.

The high surface area material can be a sol-gel derived product (Reetz et al., Biotech Bioeng 49:527-534 (1996); Frenkel-Mullerad, et al., J Amer Chem Soc 127:8077-8081 (2005), which are hereby incorporated by reference in their entirety), a hydrogel derived product such as those formed using polyacrylamide or polyethylene glycol (Xu et al., Polymer Bulletin 58(1):53-63 (2007); Gurevitch et al., JALA 6(4): 87-91 (2001); Lueking et al., Molecular & Cellular Proteomics 2:1342-1349 (2003), which are hereby incorporated by reference in their entirety), polymer brush derived product (Wittemann et al., Analytical Chem. 76(10):2813-2819 (2004), which is hereby incorporated by reference in its entirety), nitrocellulose membrane encapsulation product, or dendrimer-based products (Pathak et al., Langmuir 20(15):6075-6079 (2004), which is hereby incorporated by reference in its entirety). Of these, sol-gel derived materials are preferred.

One of the advantages of using the sol-gel material for entrapment of target molecules is that there is no need for the use of a linker or tag to immobilize the target molecule. It is highly advantageous to encapsulate target molecules in a sol-gel, because of the ease with which sol-gel materials can be miniaturized. This method is far more reliable and less cumbersome than other available methods for entrapment such as membrane encapsulation. Furthermore, entrapment in glass sol-gel materials will allow for optical monitoring of many enzymatic reactions using simple photometry. The methods for obtaining such sol-gel materials are described in detail by Wright et al., “Sol-Gel Materials: Chemistry and Applications,” CRC Press (2000); Pierre, A., “Introduction to Sol-Gel Processing (The International Series in Sol-Gel Processing: Technology & Applications),” Springer (1998); Brinker et al., “Sol-Gel Science: The Physics and Chemistry of Sol-Gel Processing,” Academic Press (1990), which are hereby incorporated by reference in their entirety.

The sol-gel process is a wet chemical process that can be used for making ceramic or glass materials. In general, the sol-gel process involves the transition of a system from a liquid “sol” (mostly colloidal) into a solid “gel” phase. Applying the sol-gel process, it is possible to fabricate ceramic or glass materials in a wide variety of forms: ultra-fine or spherical shaped powders, thin film coatings, ceramic fibers, microporous inorganic membranes, monolithic ceramics and glasses, or extremely porous aerogel materials. In accordance with the present invention, where it is desirable for the sol-gel to remain adjacent to the heating element, i.e., within the one or more chambers, coatings and monolithic structures are preferred.

The starting materials used in the preparation of the “sol” are usually inorganic metal salts or metal organic compounds such as metal alkoxides, including without limitation, those containing Si, Al, Ti, and combinations thereof. Other metal oxides can also be used. In a typical sol-gel process, the precursor is subjected to a series of hydrolysis and polymeration reactions to form a colloidal suspension, or a “sol”. Further processing of the “sol” to remove the solvent allows one to make ceramic materials in different forms of the type described above.

Sol-gel processes offer a relatively mild route for the immobilization of biomolecules such as proteins, which are entrapped in the growing covalent gel network rather than being chemically attached to an inorganic material (Gill, et al., Annals of the New York Academy of Sciences 799:697-700 (1996), Gill et al., Trends in Biotechnology 18:282-296 (2000), which are hereby incorporated by reference in their entirety). Many studies have described the encapsulation of a variety of biologicals, including enzymes, antibodies, regulatory proteins, membrane-bound receptors, nucleic acid aptamers, and even whole cells, using a wide range of sol-gel derived nanocomposite materials (Reetz et al., Biotech Bioeng 49:527-534 (1996), Frenkel-Mullerad, et al., J Amer Chem Soc 127:8077-8081 (2005), which are hereby incorporated by reference in their entirety).

With regard to stability, proteins entrapped in sol-gels typically exhibit improved resistance to thermal and chemical denaturation, and increased storage and operational stability over months or even longer (Kim, et al., J Biomat Sci 16:1521-1535 (2005), Pastor et al., J Phy Chem 111:11603-11610 (2007), which are hereby incorporated by reference in their entirety). Additionally, the dual nanoporous material of the sol-gel matrix developed by Kim et al. (Analytical Chem 78(21):7392-7396 (2006), which is hereby incorporated by reference in its entirety) can allow diffusion of molecules such as aptamers, while retaining target molecules (protein or chemicals) immobilized in the pores. This is one of the biggest advantages of sol-gel materials, which allows its applicability to SELEX methods.

In one embodiment, the molecular binding region (e.g., sol-gel with embedded target) is formed on a polymer coating. The polymer coating may be poly(meth)acrylate or PMMA (Kwon et al., Clinical Chemistry 54(2):424-428 (2008), which is hereby incorporated by reference in its entirety). This polymer coating physically separates the sol-gel material (and its embedded target molecules) from the adjacent heating electrode, which avoids direct heat exposure to the target molecules.

In an alternative embodiment of the invention, the molecular binding region(s) can include a surface of the one or more fluid channels or one or more chambers, where the surface is modified with one or more target molecules bound to the surface via a linker molecule. Microfluidic arrays can be produced on, for example, a glass or pyrex slide, which provides a flat surface. Target proteins or other target molecules are bound covalently or non-covalently to the flat surface of the solid support. The targets can be bound directly to the flat surface of the solid support, or can be attached to the solid support through a linker molecule or compound. The linker can be any molecule or compound that derivatizes the surface of the solid support to facilitate the attachment of the target to the surface of the solid support. The linker may covalently or non-covalently bind the target to the surface of the solid support. In addition, the linker can be an inorganic or organic molecule. Standard glass coupling chemistry can be employed with the linker molecules. One example of preferred linkers are compounds with free amines.

The target proteins and other targets molecules of the present invention can also be bound to a substrate (e.g., bead) that is placed and retained in the one or more chambers. Exemplary substrates include, without limitation, nitrocellulose particles, glass beads, plastic beads, magnetic particles, and latex particles. Preferably, the target molecules are covalently attached to the substrate using known procedures.

The microfluidic SELEX procedure of the present invention can be used to select aptamers that exhibit desired affinity to a wide variety of targets. For example aptamers can be identified that bind to a large molecule target, such as a protein. Exemplary large molecule targets may include, but are not limited to, IgE, Lrp, E. coli metJ protein, elastase, human immunodeficiency virus reverse transcriptase (HIV-RT), thrombin, T4 DNA polymerase, and L-selectin. Aptamers can also be identified with that bind to a small molecule target, such as a peptide, amino acid, or other small biomolecule. Exemplary small molecule targets include, but are not limited to, ATP, L-arginine, kanamycin, lividomycin, neomycin, nicotinamide (NAD), N-methylmesoporphyrin (NMM), theophylline, tobramycin, D-tryptophan, L-valine, vitamin B12, D-serine, L-serine, y-aminobutyric acid (y-ABA), and organic dyes. Aptamers may also be identified that bind to macromolecules, for example, but not limited to, viruses, such as human cytomegalovirus (HCMV), bacteria, eukaryotic cell, organelles, and nanoparticles. Broadly, suitable biological materials for use as targets include, but are not limited to a protein or polypeptide, a carbohydrate, a lipid, a pharmaceutical agent, an organic non-pharmaceutical agent, or a macromolecular complex. Carbohydrates, polysaccharides, substrates, metabolites, transition-state analogs, cofactors, drug molecules, dyes, nutrients, liposomes can also be used as targets as long as they can be immobilized within the microfluidic device, preferably within the porous sol-gel matrix. One skilled in the art can readily supplement this list of targets with other biological materials which can be used as targets of the present invention. Additionally, the biological material could be tagged or modified as desired by addition of readily detected substituents such as ions, ligands, optically active compounds or constituents commonly used to tag biological or chemical compounds.

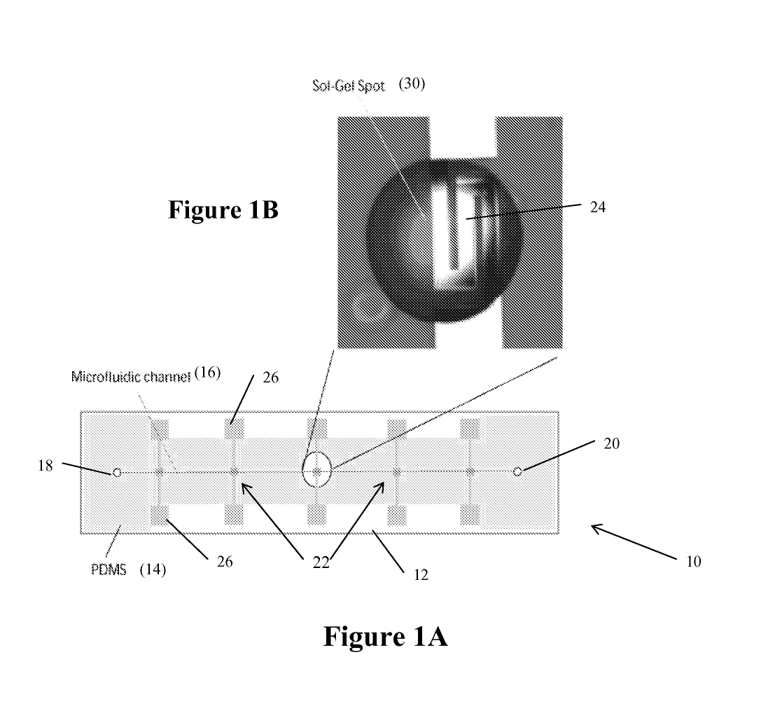

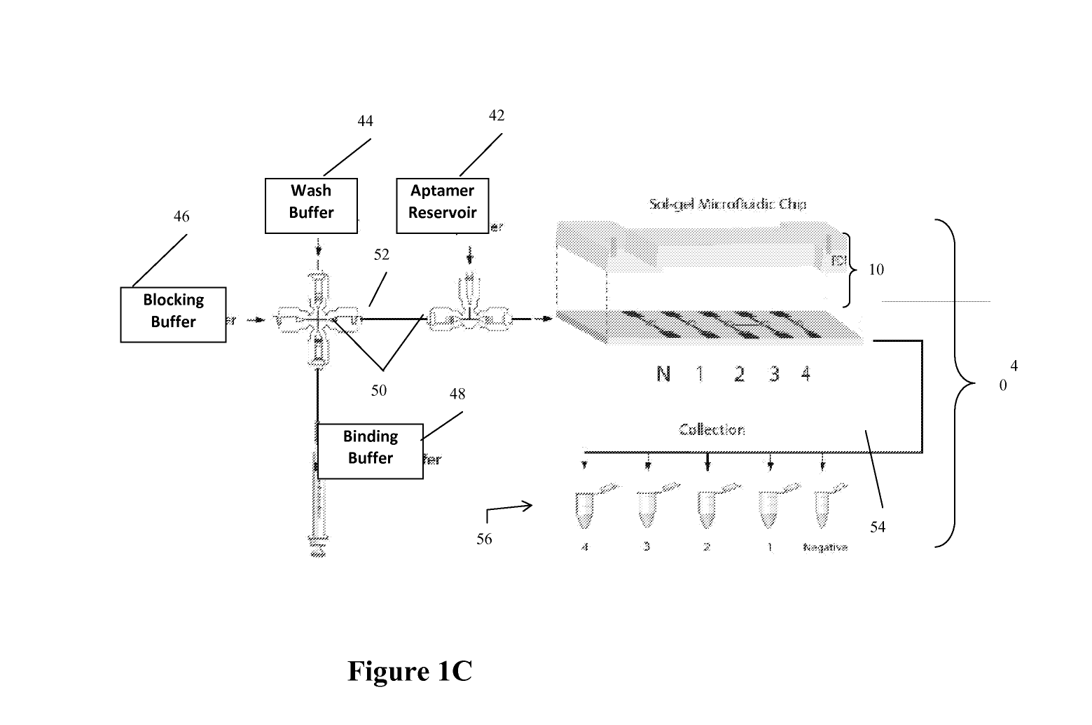

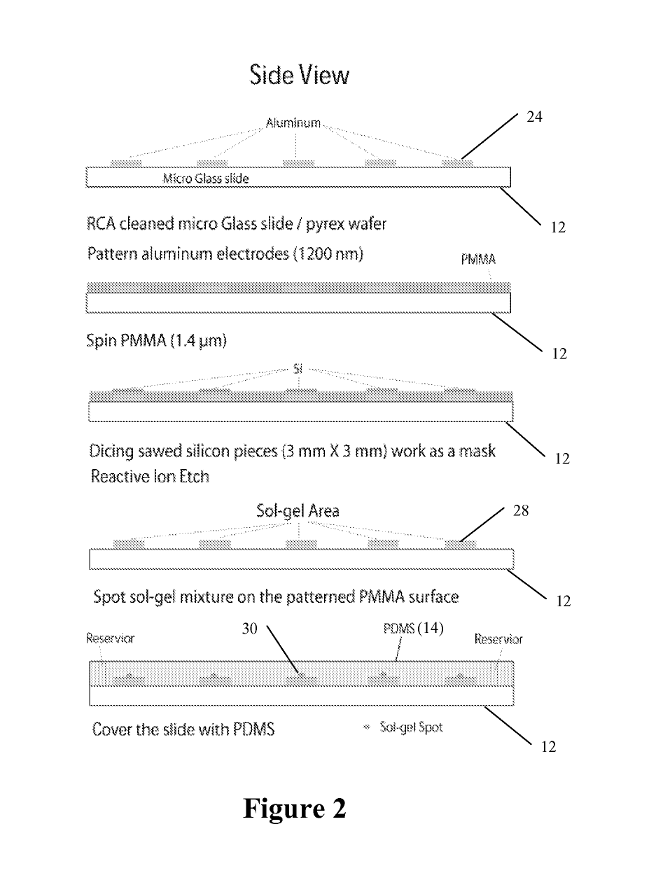

Referring now to FIGS. 1A-B and 2, one preferred embodiment of the microfluidic device 10 is illustrated. The microfluidic device 10 is formed on a glass substrate 12 with a PDMS lid 14 secured over the substrate 12. Together, the substrate 12 and lid 14 define a microfluidic channel 16 formed between in an inlet 18 and outlet 20. The channel is characterized by five chambers 22 spaced along the length of the channel 16. Each chamber 22 is positioned over a heating electrode 24, which is positioned between electrical contacts 26 on either side of the device. In this embodiment, the heating electrode 24 is physically separated from the chamber by a polymethacrylate layer 28 (see FIG. 2). Prior to securing the PDMS lid 14 over the substrate 12, a sol-gel material 30 containing a target molecule of interest is deposited into one or more of the chambers 22, preferably directly above or adjacent to the heating electrode 24 (see FIG. 1B). As described above, this effectively entraps the target molecule with the respective chamber(s) 22.

As shown in FIG. 2, fabrication of the device 10 can be carried out by first patterning one or more electrodes 24 (along with their contacts 26) onto the cleaned surface of substrate 12. The entire surface is then spin coated with a polymethacrylate polymer, masked with silicon (over the polymer-coated electrodes), and the coated substrate is etched to remove the polymer except where masked. This polymer layer 28 effectively isolates the heating electrode 24 from what will become a chamber 22 within the microfluidic device. After etching, the sol-gel containing the target molecule of interest can be formed, and the sol-gel suspension deposited onto the polymer layer 28. After deposition of the sol-gel material, the solvent is allowed to evaporate or the device is dried under appropriate conditions, thereby forming the sol-gel spot 30. Thereafter, the substrate 12 is covered with a patterned PDMS lid 14 to form the microfluidic device 10.

The microfluidic device 10 is intended to be used in combination with a microfluidic SELEX system 40, one embodiment of which is illustrated in FIG. 1C. The system 40 includes reservoirs for the aptamer population 42 (which can be an enriched pool of aptamers selected in prior rounds of SELEX or a random population of nucleic acid molecules not yet having been selected via SELEX), wash buffer 44, blocking buffer 46, and binding buffer 48. The reservoirs can be coupled together via a multiport coupling 50 and fluid lines 52 for sequential introduction of the materials into the device 10 via its inlet. Connected to the outlet is another fluid line 54, which is coupled via valves and multiport couplings, as needed, to one or more collection containers 56. Preferably, each container is intended to receive the eluted aptamer population from a single chamber of the device, i.e., which is specific for a particular target molecule. As shown in FIG. 1C, for example, containers are provided for each of the Negative Chamber and Chambers 1-4. Valves can be opened and closed to appropriately direct the eluted aptamer population from a particular chamber into its corresponding container.

Movement of the various fluids into and out of the device 10 can be controlled manually by pump, and operation of the heating elements can be controlled manually by passing current through the respective electrodes. Alternatively, the movement of fluids into and out of the device 10 and operation of the heating elements can be controlled automatically using an operating system programmed to regulate the timing of one or more pumps and one or more valves responsible for regulating the introduction of an aptamer pool, wash buffer, blocking buffer, and/or binding buffer into the device, and the timing of heating element operation for elution of bound aptamers and their subsequent collection in appropriate collection containers.

Because of the highly sequential nature of the SELEX process, various systems associated with the microfluidic chip are preferably automated and associated with software that runs on a computer and is easily programmable and modifiable. Computers in microfluidic systems of the invention can control system processes and receive signals for interpretation. For example, the computer can control a robotic sub-system that retrieves samples or analytes from storage as needed for the SELEX cycles. The computer can control specimen stations to designate the order of drawing samples and reagents for receipt into the microfluidic device. Pressure differentials and electric potentials can be applied to microfluidic devices by the computer through computer interfaces known in the art, thereby controlling pump devices and valves to regulate the flow of reagents into and out of the system. The computer can be a separate sub-system, it can be housed as an integrated part of a multi-assay instrument, or dispersed as separate computers in modular subsystems.

The computer system for controlling processes and interpreting detector signals can be any known in the art. The computer can also include a software program, which, for example, is useful for correlating, analysis and evaluation of the detector signals with the presence of one or more aptamers, evaluation of the detector signals to quantify the aptamers, detection and evaluation of power levels to calculate the amount of heat dissipated and temperatures at the heating elements, analysis of melting properties, UV absorbance calculations for the aptamers such that they can be designed, and selected. The computer can be in functional communication with the one or more valves controlling the inflow and outflow of fluids, various heating element controls in the microfluidic chip, flow rate controllers to control the rate and direction of flow inside the chip or detectors. The computer can also control power circuits, control mechanical actuators, receive the information through communication lines, store information, interpret detector signals, make correlations, etc.

Systems in the present invention can include, e.g., a digital computer with data sets and instruction sets entered into a software system to practice the multiple assay methods described herein. The computer can be a personal computer with appropriate operating systems and software control, or a simple logic device, such as an integrated circuit or processor with memory, integrated into the system. Software for interpretation of detector signals is available, or can easily be constructed by one of skill using a standard programming language such as Visualbasic, Fortran, Basic, Java, or the like.

Although the system 40 shown in FIG. 1C only includes a single chip with a single source of aptamer library, it should be appreciated by persons of skill in the art that the systems of the present invention can adapted for conducting multiple SELEX procedures by running one or more targets against two or more aptamer libraries in one or more chips. This will provide a variety of resultant aptamer-target combinations. Multi-SELEX systems can include, e.g., a microfluidic device with one or more reaction chambers holding one or more targets, two or more libraries flowing to contact targets in the one or more reaction chambers, and one or more detectors, sequencers or analytical instruments configured to detect signals resulting from the contact between the targets and the aptamer libraries. The resultant signals can be evaluated to determine the presence, sequence of aptamers or quantify the aptamers in the sample. The systems can be useful for analyzing a matrix of target/aptamer combinations.

The microfluidic chips of the present invention can be in fluidic contact with variety of specimen manipulation stations. These specimen stations can be, for example, autosamplers, such as sample carousels holding multiple aptamer libraries in a circular tray that can be rotated sequentially or randomly to align the library containers with one or more pipettors. The pipettors can be on actuated arms that can dip the pipettor tube into the specimen for sampling or delivery. The microfluidic chips can also be in communication with elution collectors which can be, for example, auto-collectors. These collectors can collect eluted fluids from various chambers of the chip during the SELEX process. Specimen stations can also be configured to hold one or more microliter plates of specimens or elutions. The station can translate the plates with an X-Y plotting motion to position any of the plate wells under a pipettor tube.

In many embodiments of the systems, the samples or reagents are of very small volume, for example, as is typical of many molecular libraries. Sampling from such libraries or eluting aptamers, e.g., on microwell plates or microarray slides, is typically accomplished with robotic systems that precisely position the pipettor tip in the micro specimen. In embodiments where the library members are retained in dehydrated form, it can be convenient to sample by ejecting a small amount of solvent from the pipettor to dissolve the specimen for receipt into the microfluidic devices of the present invention.

The methods of the present invention are directed to an improved method for SELEX using a microfluidic chip. SELEX is an “evolutionary” approach to combinatorial chemistry that uses in vitro selection to identify RNA or DNA sequences with high affinity for a particular target (Joyce, Gene, 82:83-87 (1989); Ellington et al., Nature 346:818-822 (1990); Tuerk et al., Science, 1990; 249:505-510 (1990), which are hereby incorporated by reference in their entirety). Several publications describe the SELEX process (Joyce, Curr. Opin. Struct. Biol., 4:331-336 (1994); Lorsch et al., Acc. Chem. Res., 29:103-110 (1996); Forst, J. Biotech., 64:101-118 (1998); Klug et al., Mol. Biol. Rep., 20:97-107 (1994); U.S. Pat. Nos. 5,270,163, 5,475,096, and 5,707,796 to Gold et al., which are hereby incorporated by reference in their entirety). The process separates functional high affinity molecules from random DNA or RNA pools using techniques that partition high affinity binders from low affinity binders. These functional sequences having high affinity towards targets have found use as drugs that act on specific biological receptors or as diagnostic agents that can be used in biomedical analyses or imaging.

The general procedure for conventional SELEX involves screening a pool of randomly sequenced DNA is generated (approximately ≧10¹⁴-10¹⁵ independent sequences). Often the DNA is transcribed to RNA, which has been shown to be more functional than DNA. The RNA pool is then passed through a chamber with the target molecule attached to a stationary phase. RNAs with affinity towards the immobilized target molecule are retained on the stationary phase. RNAs with little or no affinity for the target molecule are washed off. The bound RNAs are then eluted off the stationary phase using a solution containing the free ligand, or by changing the binding conditions. The eluted RNA molecules are then reverse transcribed, with the resulting DNA being amplified using PCR. When repeated several times, the selection cycle eliminates the inactive RNAs from the pool, leaving only sequences with specific and high affinity for the target molecule. The SELEX process has been successful in selecting molecules that have affinity for various target molecules (Wiegand et al., J. Immun., 1996; 157:221-230 (1996); Huizenga et al., Biochem., 1995; 34: 656-665 (1995), which are hereby incorporated by reference in their entirety).

The microfluidic SELEX selection procedure of the present invention is used to identify nucleic acid ligands of a target molecule from a candidate mixture of nucleic acids using a microfluidic chip. Such a candidate mixture of nucleic acids may also be referred to as “a library,” “a combinatorial library,” “a random combinatorial library,” a “combinatorial pool,” a “random pool,” or a “randomized DNA pool.” By way of example, in a candidate mixture of nucleic acids that is to be screened, each nucleic acid sequence can have a random region flanked by two primer-specific regions. The number of random nucleotides can be any size, but typically between 10 and 80 nucleotides in length, more preferably 20-60 nucleotides in length. The primer-specific regions can also be any size that allows them to function as primers, but typically they are between 10-40 nucleotides in length, preferably about 15-30 nucleotides in length. Regardless, the number of nucleotides in the random region can be easily increased or decreased as desired. Similarly, the primer sequences on each end can be modified according to the condition required for PCR. Further, the primer sequences used in a library may be chosen to minimize primer-primer interactions or the formation of primer dimers during PCR. Primers may be designed with a variety of melting temperatures; methods of designing primers are well known in the art.

Such a pool includes nucleic acid molecules that will exhibit affinity to the target molecule as well as nucleic acid molecules that will not. The candidate mixtures of nucleic acids may be randomized pools of single stranded DNA or single stranded RNA. The libraries used for microfluidic SELEX may also be similar to the randomized pools of DNA or RNA used in conventional SELEX (He et al., J Mol Biol 255:55-66 (1996); Bock et al., Nature 355:564-566 (1992), which are hereby incorporated by reference in their entirety). Alternatively, the pool used for introduction into the microfluidic SELEX process can be a partially selected pool that has been passed through conventional SELEX for one or up to several rounds.

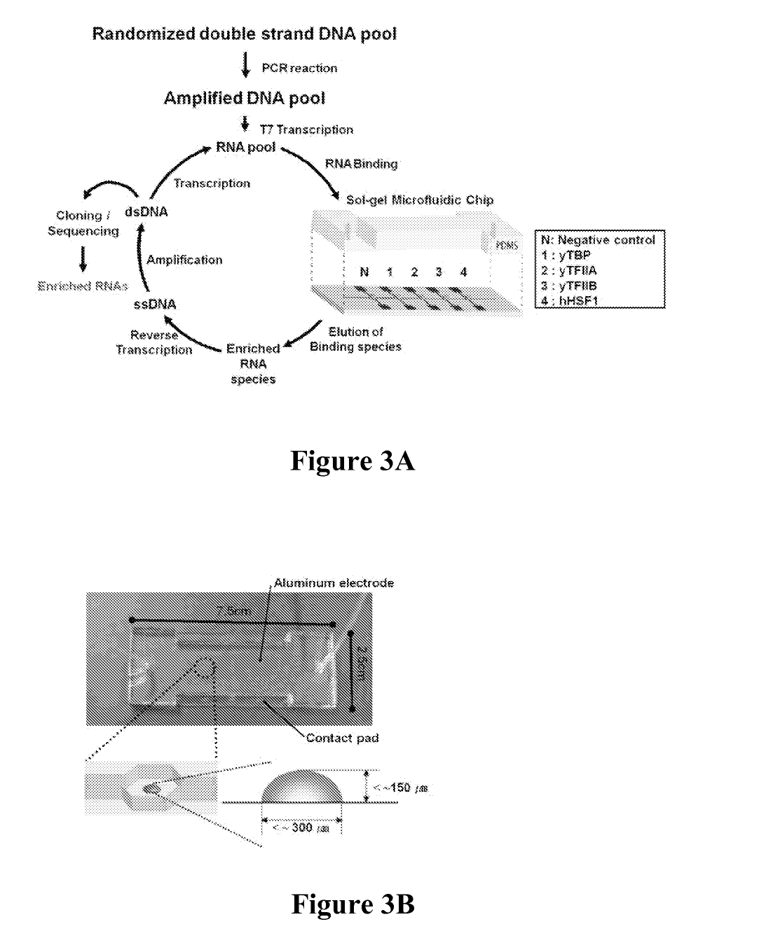

Referring to FIG. 3A, the microfluidic SELEX process is directed to selecting a nucleic acid aptamer for binding to one or more target molecules. The method includes introducing a nucleic acid population into the microfluidic device under conditions effective to allow the nucleic acid molecules to bind specifically to the target molecule. The method further includes removing from the microfluidic device substantially all nucleic acid molecules that do not bind specifically to the target molecule, thereafter heating the heating element to cause denaturation of nucleic acid molecules (i.e., aptamers) that bind specifically to the target molecule, and then recovering nucleic acid molecules that bind specifically to the target molecule. The recovered nucleic acid molecule, which are aptamers, have been selected for their binding to the target molecule.

The process can be repeated any number of times. For example, the resulting enriched population of target-binding nucleic acid molecules can be reverse-transcribed (as needed), amplified, and then either (i) cloned and sequenced, (ii) transcribed to form an enriched pool of target-binding nucleic acids that can be passed through a microfluidic SELEX device loaded with the same target molecule; or both.

The microfluidic SELEX process of the present invention yields a class of products that are referred to as aptamers, each having a unique sequence. As used herein “aptamers” are nucleic acid ligands that have the property of binding specifically to a target compound or molecule. However, the term “aptamer” does not quantify the affinity of the nucleic acid to the target. For the purposes of the present invention, aptamers with high affinity to targets are selected from a pool of lower affinity aptamers. Thus, aptamers can have a high binding affinity for a target and exhibit molecular recognition. The selected aptamers may be cloned and sequenced, allowing the production of large quantities of a single isolated and purified aptamer.

In one embodiment of the invention, the nucleic acid aptamers are formed of RNA, and the method further comprises performing reverse transcription amplification of the selected aptamer population. The selected RNA aptamers obtained after the microfluidic SELEX process can be reverse transcribed to DNA using standard reverse transcription techniques known in the art. Reverse transcription is a method of enzymatically converting a single stranded RNA sequence into a single stranded DNA sequence. The enzymes used for reverse transcription are known as RNA dependent DNA polymerases (U.S. Pat. Nos. 5,322,770 and 5,641,864 to Gelfand; U.S. Pat. No. 6,013,488 to Hayashizaki, which are hereby incorporated by reference in their entirety).

In another embodiment of the invention, the nucleic acid aptamers are formed of DNA, in which case the enriched pool of aptamers can be amplified directly, cloned and sequenced as desired, and re-introduced through a microfluidic SELEX device loaded with the same target molecule.

The method can further include purifying and sequencing the amplified aptamer population. The resultant amplified aptamer obtained with the microfluidic SELEX procedure is still a mixture of aptamer sequences with similar binding affinities toward the target molecule. These differences may be minor (for example, a similar sequence appearing at a different position on the aptamer) or may represent completely different binding mechanisms (binding to different sited on the target molecule). Cloning and sequencing may be used to characterize individual aptamers, and to facilitate the identification of binding motifs. Any of the various cloning and sequencing procedures known to those of skill in the art may be used for the characterization of individual aptamers.

The microfluidic devices can be designed to have chambers and channels in fluid contact with a chamber that contains the target molecule, such that the aptamers and reagents mixed together can come into contact with target molecule(s) to form a reaction mixture that may or may not generate a specific signal. Target chambers can be in fluid contact with the reagents in other chambers of the microfluidic device, or preferably in fluid contact with reagents or aptamer library pool or fluid manipulation stations which receive or deliver multiple reagents, reactants, or products in series or in parallel.

Reagents can be any composition useful in the SELEX process, for example, chemicals or biomolecules capable of interacting with aptamers or target molecules, controlling the reaction conditions, or generating a detectable signal. Reagents are typically one or more molecules in a solution or immobilized on the microfluidic chip that can flow into contact with the target in a chamber or come in contact with the aptamers or the aptamer pool. For example, reagents can be wash buffers, binding buffers, or blocking buffers. Other reagents include a chromophore that reacts with the target to provide a changed optical signal. Reagents in the systems can also include molecules attached to media (e.g., a gel or solid support) and capable of interacting with targets or aptamers. For example, the reagent can be an affinity molecule on a solid support. More than one reagent can be involved in generating a detectable signal. Typical reagents on the systems of the present invention include, for example, a locus specific reagent, a PCR primer, a labeled ligand, a chromophore, an antibody, a fluorophore, an enzyme, a fluorescent resonant energy transfer (FRET) probe, a molecular beacon, a radionuclide, and/or the like.

Within the microfluidic devices are chambers where target molecules come into contact with specific reagents and aptamers. These chambers can also be configured to provide conditions necessary to provide a detectable signal resulting from the contact between targets and aptamers or to provide conditions for partitioning of specific or higher affinity aptamers from non specific or lower affinity aptamers. The affinity of aptamers to the target molecules will depend on each individual target, reaction conditions, and the aptamer pool used. For example, reaction chambers can receive forces to induce flows, have controlled temperatures to lead to binding or to elution, have sufficient lengths to provide adequate incubation times during flow, have solid supports to hold or capture reaction constituents, hold selective media, and/or the like. The devices can have a single reaction chamber or multiple reaction chambers.

Reaction chambers can also be, for example, thermocycler amplification chambers that cycle through a programmable temperature profile a number of times while the reaction mixture is present in the chamber. Amplification reactions in thermocycling chambers are typically polymerase chain reactions (PCR) to amplify rare or dilute nucleic acid sequences from a sample so they can be detected or sequenced. A number of high throughput approaches to performing PCR and other amplification reactions have been developed, for example, involving amplification reactions in microfluidic devices, as well as methods for detecting and analyzing amplified nucleic acids in or on the devices (U.S. Pat. No. 6,444,461 to Knapp, et al.; U.S. Pat. No. 6,406,893 to Knapp, et al.; U.S. Pat. No. 6,391,622 to Knapp, et al.; U.S. Pat. No. 6,303,343 to Kopf-Sill; U.S. Pat. No. 6,171,850 to Nagle, et al.; U.S. Pat. No. 5,939,291 to Loewy, et al.; U.S. Pat. No. 5,955,029 to Wilding, et al.; U.S. Pat. No. 5,965,410 to Chow, et al.; Zhang et al. Anal Chem. 71:1138-1145 (1999), which are hereby incorporated by reference in their entirety).

In some cases, reaction chambers can also act as incubators and/or mixers of various reagents, e.g., for a chemical or biomolecules to specifically react with the target to generate a binding configuration. In other cases, reaction chambers can include reagents in the form of selective media. Selective media can be those known in the art, such as, size selective media (e.g., size exclusion media or electrophoresis gels), ampholyte buffers used in isoelectric focusing (IEF) techniques, ion exchange media, affinity media (e.g., lectin resins, antibodies attached to solid supports, metal ion resins, etc.), hydrophobic interaction resins, chelator resins, and/or the like. For example, contact of a sample with a size exclusion media reagent can resolve a nucleic acid aptamer of interest from other constituents so that an absorbance signal after a predetermined retention time can be interpreted to determine the presence or quantity of the nucleic acid in the sample.

Microfluidic devices can also have detection regions that can be monitored by detectors which detect the signals, for example, resulting from contact of targets with aptamers, a signal from a reagent that has reacted with a sample analyte, the absence of a detectable signal (interpretable, e.g., as the absence of sample analyte at a level adequate to generate a signal above the sensitivity of the detector), a signal amplitude related to a quantity of a sample analyte, and/or the like. The detection regions can be one or more channels, chamber segments, or chambers in functional contact with sensors. For example, detector regions can incorporate sensors such as pH electrodes, conductivity meter electrodes. Detection regions can comprise one or more chambers transparent to certain light wavelengths so that light signals, such as, absorbance, fluorescent emissions, chemoluminescence, and the like, can be detected. Detectors can be located in the microfluidic device, or proximate to the device, in an orientation to receive signals resulting from the sample contact with the reagent. Detectors can include, e.g., a nucleic acid sequencer, a fluorometer, a charge coupled device, a laser, a photo multiplier tube, a spectrophotometer, scanning detector, microscope, or a galvo-scanner. Signals detected from interactions of reagents and samples can be, e.g., absorbance of light wavelengths, light emissions, radioactivity, conductivity, refraction of light, etc. The character of signals, such as, e.g., the amplitude, frequency, duration, counts, and the like, can be detected.

Detectors can detect signals from detector regions described by physical dimensions, such as a point, a line, a surface, or a volume from which a signal can emanate. In many embodiments, the detector monitors a detection region that is essentially the point along a channel where a reaction mixture flows out from a reaction channel. In other embodiments, the detector can scan a detection region along the length of a channel while the reaction mixture is flowing or stopped. In still other embodiments, the detector can scan an image of a surface or volume for signals resulting from interactions of reagents and samples. For example, a detector can contemporaneously image multiple parallel channels carrying reaction mixtures from multiple analyses to detect results of several different assays at once.

The detectors can transmit detector signals that express characteristics of resultant signals received, For example, the detector can be in communication with an output device, such as an analog or digital gage, that displays a value proportional to a resultant signal intensity. The detector can be in communication with a computer through a data transmission line to transmit analog or digital detector signals for display, storage, evaluation, correlation, and the like.

Although in the depicted embodiments described above, a heating element is used to denature the nucleic acid bound to a target molecule. In an alternative embodiment of the invention, the microfluidic device can be modified to omit the heating element and instead include a reservoir that contains a high stringency wash agent that effectively causes chemical denaturation of the nucleic acid aptamers. Denaturation is a process in which nucleic acids lose their tertiary and secondary structure by application of some external stress or chemical, such as a strong base or a chaotropic agent like formamide, ganidinium, or urea. The denaturation of nucleic acids such as DNA or RNA often also occurs due to high temperatures. At the secondary structure level, the denaturation is the separation of a double strand into two single strands. This occurs when the hydrogen bonds between the strands are broken. At the tertiary structure level the interactions, such as hydrogen bonding, between various parts of RNA may be disrupted by denaturants.

The methods of the present invention may be such that the recovering, performing reverse transcription, amplification, purifying, and/or said sequencing are performed in one or more separate fluidic devices coupled in fluid communication with the microfluidic device of the present invention. These devices can be, for example, thermocycler amplification chambers, chromatographic chambers, incubation chambers, affinity capture chambers, sequence detection chambers or devices performing similar tasks. The chambers can also include detection regions or lead into detection regions for detection of resultant signals from, for example, sequencing reactions. Resultant signals can be detected by any appropriate detector. The detector can separately detect signals from two or more of the reaction chambers in series or in parallel. The resultant signals providing information about aptamers or targets or other analytes in the samples can be, for example, detectable signals from reagents that have reacted with sample aptamers or signals from the binding of aptamers to the targets, a lack of a detectable signal, and/or a signal amplitude related to a quantity of a aptamers binding to the targets. The detector can be, for example, a fluorometer, a charge coupled device, a laser, an enzyme, an enzyme substrate, a photo multiplier tube, a spectrophotometer, scanning detector, microscope, a galvo-scanner, a mass spectrometer, Liquid Chromatography-Mass Spectrometer, High Pressure Liquid Chromatography (HPLC) or other chromatographic detection methods, and/or the like.

The aptamers of the present invention will be useful as tools in analytical chemistry, useful in a wide range of diagnostic assays and will have direct benefits to many areas of research, including biomedical and health research. For example, increased binding efficiency and and/or increased binding selectivity will be beneficial in developing aptamer drugs that act on specific biological receptors. Aptamers with improved binding efficiency and selectivity will demonstrate increased pharmacological activity with fewer side effects. Improved aptamers will also be useful in developing diagnostic assays where detection limits are often related to binding affinity. Improved aptamers will also find use in many areas as diagnostic markers in, for example, medical analyses, in vivo imaging and biosensors. Improvements in selectivity will also be advantageous in quantization of targets present in complex matrices. Aptamers may be developed for use in other aptamer-based assays, such as assays for analytes. Various ways of using the aptamers are described in the prior art and the methods disclosed in the present invention can readily be extended to such applications (German et al. Anal. Chem., 70:4540-4545 (1998); Jhaveri et al., J. Amer. Chem. Soc., 122:2469-2473 (2000); Lee et al., Anal. Biochem., 282:142-146 (2000); Bruno et al., Biosens. Bioelec., 14:457-464 (1999); Blank et al., J. Biol. Chem., 279:16464-16468 (2001); Stojanovic et al., J. Am. Chem. Soc., 123:4928-4931 (2001), which are hereby incorporated by reference in their entirety).

The microfluidic SELEX process of the present invention may also be used to develop diagnostic assays for compounds of neurological interest—such as neuropeptides or small molecule neuromessengers, such as glutamate and zinc. Aptamer based diagnostic assays will also facilitate the analysis of neuropeptides, which are often present at picomolar concentrations in vivo. Aptamers may be used as drugs, designed by selecting for molecules with affinity for certain biological receptors (Osborne et al., Chem. Rev., 97:349-370 (1997); Brody et al., Rev. Mol. Biotech., 74:5-13 (2000); White et al., J. Clin. Invest., 106:929-934 (2000), which are hereby incorporated by reference in its entirety). Such aptamer drugs can be used to modify biological pathways or target pathogens, such as viruses or cancerous cells, for elimination. For example, aptamers that bind IgE inhibit immune response and may be useful in treating allergic reactions and asthma (Wiegand et al., J. Immun., 1996; 157: 221-230 (1996), which is hereby incorporated by reference in its entirety). The SELEX method of the present invention may also be used in the selection of RNAs or DNAs that not only bind a target molecule, but also act as catalysts (Lorsch et al., Acc. Chem. Res., 29:103-110 (1996), which is hereby incorporated by reference in its entirety). Aptamers of the present invention includes aptamers containing modified nucleotides conferring improved characteristics on the nucleic acid ligand, such as improved in vivo stability or improved delivery characteristics. Examples of such modifications include, but are not limited to, chemical substitutions at the ribose and/or phosphate and/or base positions.

A further aspect of the present invention relates to kits that include a microfluidic device or chip of the present invention, and Optionally one or more of a random pool of nucleic acid molecules, wash buffer, binding buffer, blocking buffer, reagents for carrying out reverse transcription, PCR, and/or transcription, as well as directions for carrying out the microfluidic SELEX processes described herein. The microfluidic device or chip of the present invention can be provided in the kit in a fully assembled form, in which case the device is pre-loaded with one or more target molecules in distinct chambers. Alternatively, the microfluidic device or chip can be provided in an unassembled form, in which case the kit can also contains reagents for immobilizing the target molecule, preferably reagents for forming a high surface area material (e.g., sol-gel reagents) and instructions for carrying out the immobilization and assembly of the device or chip.

### EXAMPLES

The invention will be further clarified by the following examples which are intended to be exemplary of the invention.

### Materials and Methods for Example 1-8

Chemicals and Materials: SU-8 2075 and PMMA A11 were purchased from Microchem (Newton, Mass.). Plain glass slides were acquired from VWR (Batavia, Ill.). Pyrex wafers with a 4-inch diameter for multi-chip fabrication were provided by Coming (Coming, N.Y.). The recombinant yeast TATA-binding protein (TBP) and yeast TFIIB (Transcription Factor IIB) proteins were prepared as described (Fan et al., Proc Nat'l Acad Sci USA 101:6934-6939 (2004), which is hereby incorporated by reference in its entirety). SDS-PAGE gel electrophoresis was used to confirm the expression of the proteins. To prepare a SDS-PAGE gel, 2 ml of 30% acrylamide mixture, 1.25 ml of 1.5 M Tris buffer (pH 8.8), 1.7 ml of deionized water, 100 μl of 10% SDS and 100 μL of 10% APS were mixed so that the final concentration of acrylamide gel was 12%. A Sylgard 184 silicone elastomer kit for PDMS fabrication was obtained from Dow Coming Corporation (Midland, Mich.). All capillary supplies including a lure lock, capillaries and connectors were obtained from Upchurch Scientific (Oak Harbor, Wash.). 50 μl and 25 μl syringes for injecting RNA aptamers were purchased from Hamilton (Reno, Nev.). Syringes (1 ml and 3 ml) for flowing buffers to the microfluidic device were acquired from Aria Medical (San Antonio, Tex.).

Protein Preparation: Full length His-tagged versions of yeast TBP (TATA binding protein), TFIIB (Transcription Factor II), and hHSF1 (human Heat Shock Transcription Factor 1) were purified from BL21-DE3 cells according to a standard His-tagged protein purification protocol (Fan et al., Proc. Nat'l. Acad. Sci. USA 101:6934-6939 (2004); Sevilimedu et al., Nucleic Acids Res. 36:3118-3127 (2008); Zhao et al., Nucleic Acids Res. 34:3755-3761 (2006), which are hereby incorporated by reference in their entirety). In the case of yeast TFIIA (Transcription Factor IIA), recombinant proteins were purified by using a protocol obtained from S. Hahn (Fred Hutchinson Cancer Research Center, Seattle), in which subunits Toa1 and Toa2 were expressed separately in E. coli, denatured in 8 M urea, combined and renatured by dialyzing out the urea (Hahn et al., Cell, 58:1173-1181 (1989), which is hereby incorporated by reference in its entirety). Dialysis membrane (MW 10,000) was prepared as directed by the manufacturer. The purified target protein fractions were dialyzed overnight at 4° C. against 1 L dialysis buffer (20 mM Tris-HCl, 50 mM KCl, and 10% glycerol, pH 8.0). The expression and purification of these proteins were confirmed by SDS-PAGE.

### Example 1

### Fabrication of Microfluidic Device for SELEX-on-a-Chip

A microfluidic chip of the type illustrated in FIGS. 1A-B includes a PDMS (polydimethylsiloxane, Dow Corning, Mich.) lid with a microfluidic channel or chambers; and a glass or Pyrex slide with a set of aluminum electrodes. A Sylgard 184 kit provided a curing agent and a silicone elastomer base for manufacturing PDMS lids. A (1:10 w/w) ratio of curing agent to elastomer base yields good performance and elasticity of the PDMS lid. After mixing the curing agent and elastomer base and degassing the mixture, this mixture was poured against a premade SU-8 (SU-8 2075, Microchem) master. This SU-8 master was patterned on a 1 mm thick silicon wafer using standard optical lithography. The microfluidic parts embossed on the PDMS lid were 170 μm deep and 300 μm wide microchannels and five hexagonal chambers or wells with a side length of 1 mm. The thickness of the PDMS lid was about 5 mm (see FIG. 3B).

Aluminum was selected as a heater metal, because its ductility allows stress-free deposition of over a micron thick layer. Although both a plain glass slide and a 4 inch Pyrex wafer have been used as a substrate material for depositing aluminum (and a greater number of electrode assemblies can be introduced onto the Pyrex wafer), the device prepared for SELEX-on-a-chip utilized a plain glass slide patterned with 5 electrode assemblies. The glass slide was cleansed using the RCA clean method. The RCA clean method includes a first step, which is performed with a 1:1:5 solution of NH₄OH, H₂O₂, and H₂O at 75° C.; and a second step, which is performed with a 1:1:6 solution of HCl, H₂O₂, and H₂O at 75° C. This procedure eliminated the organic contaminants on the surface of the glass slides. The glass slide was then covered with a photoresist. Standard photolithography was used to pattern the photoresist layer. Aluminum was then deposited onto the surface of the photoresist layer. Using an electron beam evaporator, (Evaporator-CHA MARK 50), an aluminum layer with a total thickness of 1.2 μm was obtained. After deposition, the photoresist was removed gradually by N-methyl pyrollidone, a lift-off solvent (Microposit 1165, Microchem), over a 24 hour period. The resulting electrodes work as a localized heat source for releasing the bound aptamer from a selected element of a protein binding array. This is illustrated in FIG. 2.

After deposition of the aluminum electrode on the glass slide surface, the 1.4 μm thick polymethyl-methacrylate (PMMA) layer was patterned using standard photolithography and a reactive ion etch process using Plasma Therm 72 (Qualtx Technology Inc., Tex.). This is also illustrated in FIG. 2.

Before bonding of the PDMS lid, a sot-gel mixture containing a target protein was deposited onto the patterned PMMA surface on top of an aluminum electrode (FIGS. 1B and 2). Sol-gel materials were prepared according to the method described previously (Kim, et al., J. Biomat. Sci. 16:1521-1535 (2005), which is hereby incorporated by reference in its entirety), with minor modifications.

For the device of Examples 2-4 below, only TBP was loaded into the sol-gel. For the device of Example 5, only TFIIB was loaded into the sol-gel.

For the device of Example 6, the sol-gel droplets containing the proteins yTBP, yTFIIA, yTFIIB and hHSF1 were separately spotted on the center of a single Al-electrode heater using a pin-type spotter (Stealth Solid pin, SNS6). These four protein-loaded sol-gels were located in chambers 1-4, as indicated in FIG. 3A. The fifth chamber, N, was maintained as a negative control and was loaded with no protein. For the gelation, the chip remained in a humidity chamber (˜80% humidity) for over 12 hours. The patterned PMMA layer enhances the attachment of the sol-gel networks to the surface; furthermore it protects the aluminum layer from possible electrochemical etching while it is under electrical contact. The spots of silicate sol-gel networks were approximately 300 μm in diameter, resulting in a typical volume of about 7 nl.

After completion of gelation, a conducting gold layer (around 20 nm) was deposited on the surface of sol-gel spot using a lift-off deposition process. A electron beam evaporator (Evaporator-CHA MARK 50) was used. Then, the surface of the sol-gel spot was observed using scanning electron microscope (Zeiss Ultra, Carl Zeiss, Germany).

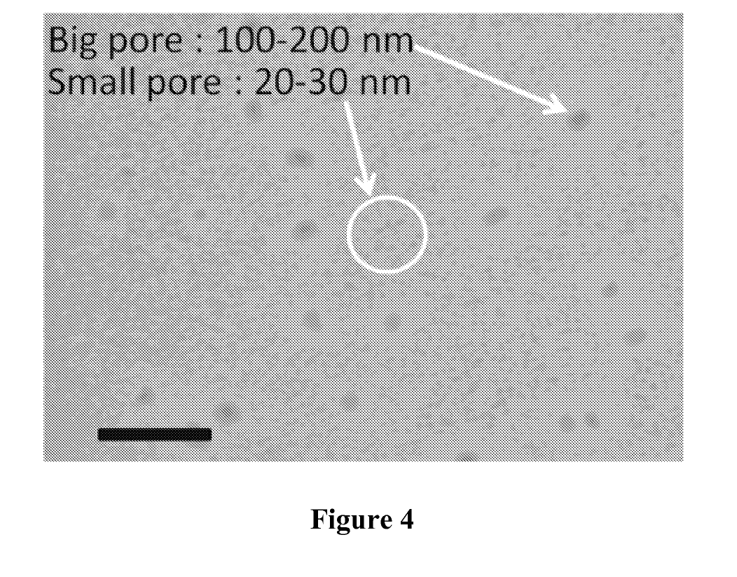

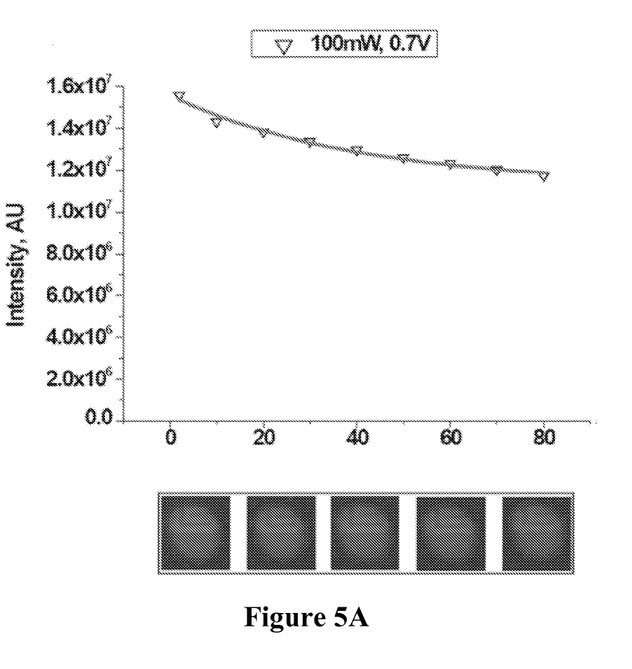

Two different types of pores were observed. Small size pores were approximately 20-30 nm in diameter, and large size pores were around 100-200 nm in diameter (FIG. 4). These pores, which are evenly distributed over the whole sol-gel surface, work as molecular passages to immobilized proteins inside. Five sets of sol-gels were spotted evenly along the microfluidic channels.

The distance between the sol-gels, based on placement of the chambers and electrodes, was kept at 1 cm to prevent unwanted heating of buffer by the other electrodes. For incubation and reaction purposes, a hexagonal chamber was placed around the sol-gels. The volume of this hexagonal chamber and the connecting channel between the chambers were 0.22 μl and 0.4 μl, respectively.

While the sol-gel was in the gelation stage, PDMS was cast on the SU-8 master. Before bonding, the sol-gel spots were protected from oxygen plasma damage by covering them with PDMS cell culture wells and their plastic lids (Culture well, Grace Bio-Lab). The glass substrates and the PDMS lid were bonded under oxygen plasma treatment. FIG. 2 shows a schematic diagram of the microchip fabrication procedure in detail. The completed microfluidic device and the experimental set up are shown in FIGS. 1A-C and 3B. The finished dimension of the microfluidic chip was 75 mm×25 mm×5 mm.

### Example 2

### Heater Electrode Design and Characterization

Sets of five heater electrodes were integrated into the microfluidic chip as described above. These electrodes contained two pad areas for probe station use and a narrow resistor area for generating heat. The total resistance of the electrode was about 2<5Ω depending on its thickness. To characterize the heater electrode, sol-gels containing TBP and TATA DNA with a known melting temperature of 81.5° C. were heated under varying conditions. The yeast TATA binding protein (TBP) and the TATA DNA region as a protein-aptamer pair was used, because TBP is a well-defined test system. TBP recognizes the most important eukaryotic core promoter motifs, the TATA element. TBP is mandatory for transcription by all RNA polymerases in yeast. TBP and intercalating SYBR Green™ (Invitrogen, Molecular Probes) dye labeled TATA DNA were incorporated into a mixture while the sol-gels were in preparation. The TATA DNA melting temperature was determined using a quantification PCR machine After complete gelation, the sol-gels in the microfluidic chambers, with a 90 μL/min flow of a binding buffer, were heated by applying currents to the electrodes using the Keithley 2400 source meter (Cleveland, Ohio), which yields power up to 22 W. The effect of heating was simultaneously observed under a fluorescence microscope. IP-Lab software was used to take 30 consecutive fluorescent images over 5 minutes. The fluorescent intensity of each sol-gel spot was analyzed using a Matlab designed program.

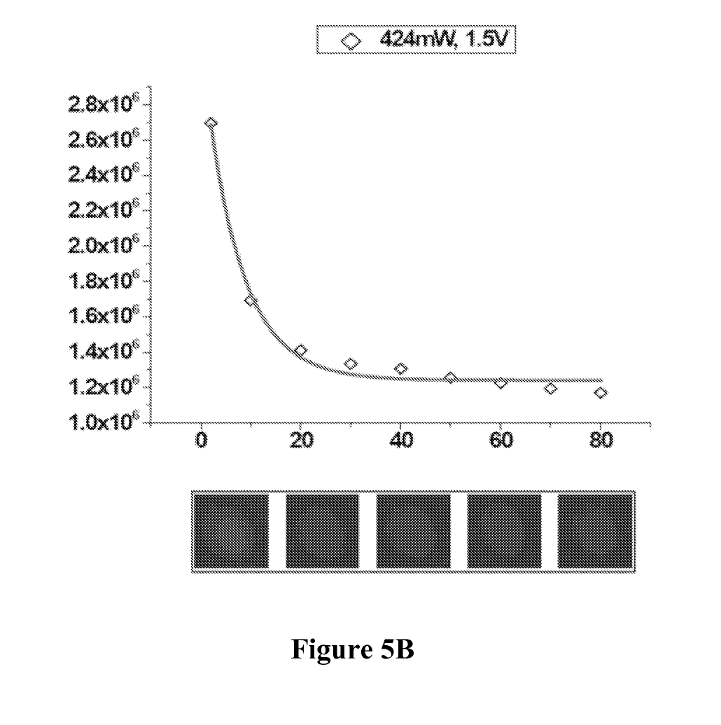

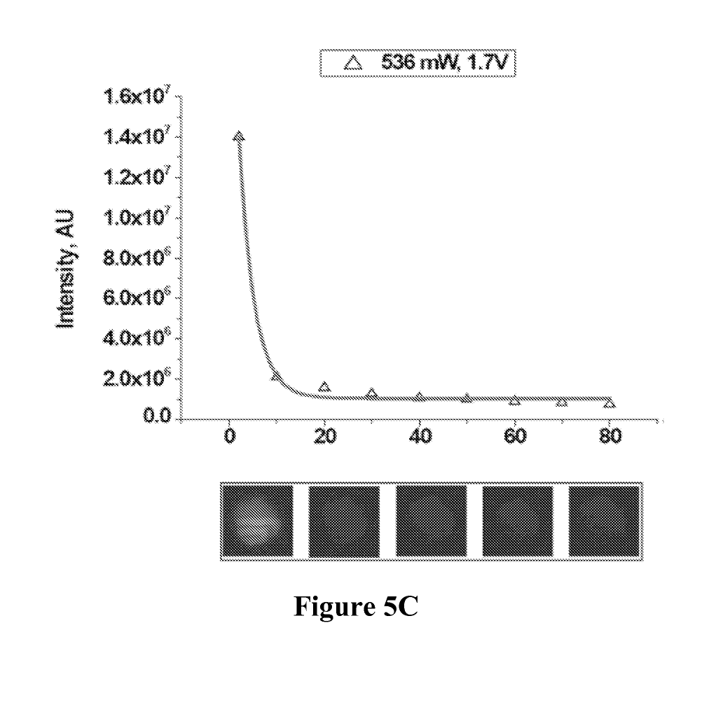

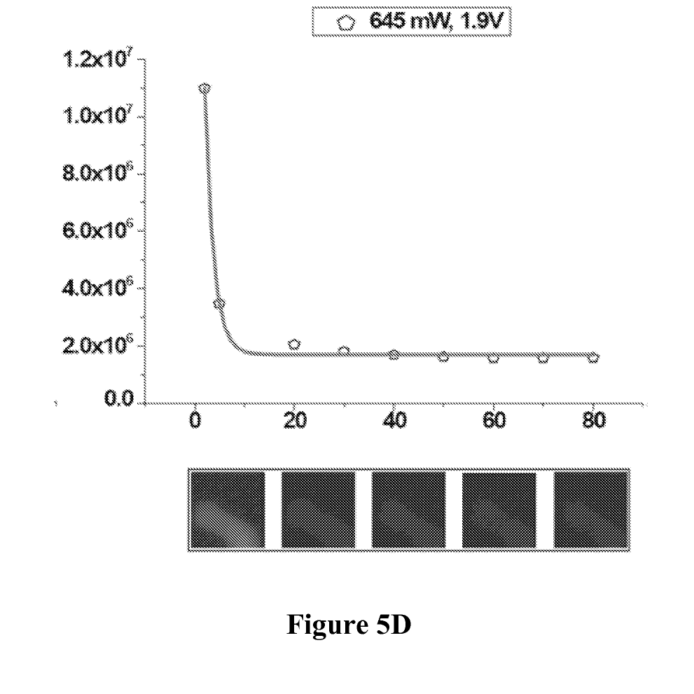

As shown in FIGS. 5A-D, various electric potentials were applied to the electrode. The corresponding fluorescent images of sol-gels were attached to each graph. Independently, the ability of the electrode to boil the PBS buffer droplet (<10 μl) within 2 minutes was confirmed. Based on this, electric power of 100 mW, 424 mW, 536 mW, 645 mW have been delivered to the individual sol-gel. Consecutive fluorescent images (20 seconds gap between the images) were taken while the electric power was being delivered and the intensities of each image were plotted against time. The behaviors of these intensities seemed to obey the exponential decay model. Therefore, the data was fit to the model:

I=I_{B}+I₀e^{-t/τ}

where I is the intensity of the sol-gel, I_{B} is the intensity of the nonspecific bindings of fluorescent molecules to the sol-gel, I₀ is the initial intensity of the sol-gel and τ indicates the half-life time of the intensity. All four graphs show good agreement between the obtained data and fit the model from the above equation with a high correlation (R²<0.9853, 0.9905, 0.9969, 0.9976 for 100, 424, 536, 645 mW, respectively). Also, the half-life time for the intensity decay was around 39.4 sec, 7.4 sec, 3.4 sec and 1.8 sec for 100 mW, 424 mW, 536 mW and 645 mW, respectively. These results indicate that aluminum electrodes heated the sol-gel above the melting temperature of the TATA DNA (81.5° C.) when power above 400 mW was delivered to the electrodes.

### Example 3

### Visualization of the Interaction Immobilized Proteins and Nucleic Acid

Sol-gels with TBP were enclosed with the PDMS lid. After encapsulation, the channels were washed extensively with the PBS buffer (binding buffer) by connecting one end of the main channel to a syringe pump (Pump 11, Harvard Apparatus, Holliston, Mass.). Following the pre-washing step, the silicate gel spot was blocked for 1 hour with 1× binding buffer that contained 25 mM Tris-Cl (pH 8), 100 mM NaCl, 25 mM KCl and 10 mM MgCl₂ with 5% skim milk. The blocking buffer works as a nonspecific competitor in the reaction mixture, which helps to achieve the selection of high-affinity molecules. Then synthetic complementary TATA DNA, with nucleic acid sequences of 5′-Cy3-GGGAA TTCGG GCTAT AAAAG GGGGA TCCGG-3′ (SEQ ID NO: 1) and 5′-CCGGA TCCCC CTTTT ATAGC CCGAA TTCCC-3′ (200 pmole) (SEQ ID NO: 2), were mixed in annealing buffer (20 mM Tris-Cl (pH 7.5), 10 mM MgCl₂, and 50 mM NaCl), with the final volume, 50 μl, incubated 5 minutes at 95° C., and then cooled slowly down to room temperature. Cy-3, a cyanine dye, was conventionally used to measure the melting temperature of the double stranded DNA. This Cy-3 labeled TATA DNA was introduced to the microfluidic chambers, and the DNA was incubated for 2 hours. A washing step with the wash buffer followed. After binding, the interaction of immobilized TBP proteins and Cy-3 labeled TATA DNA was monitored by fluorescence microscopy.

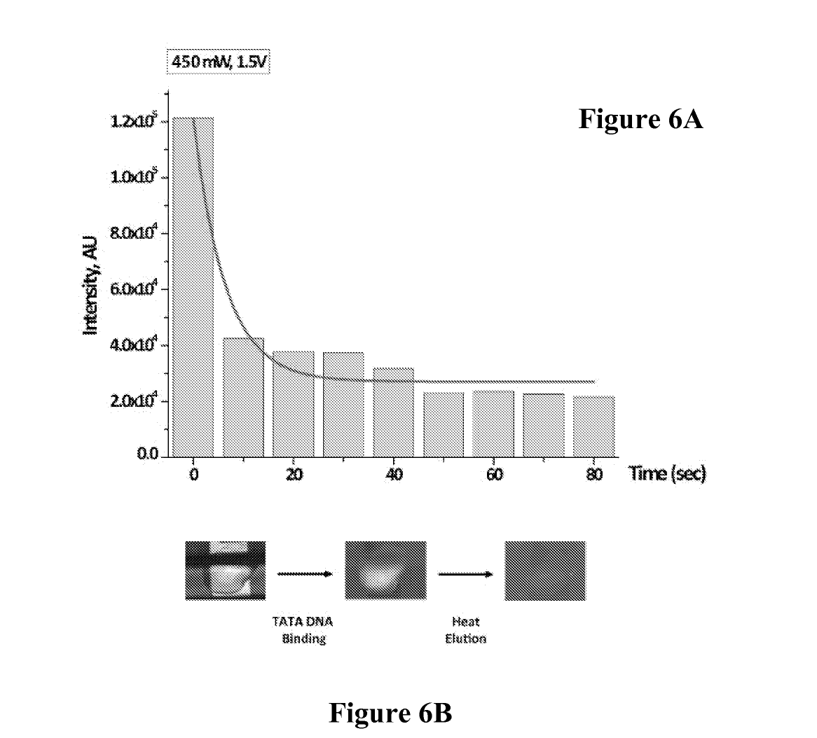

The Cy-3-labeled TATA DNA has a high affinity for TBP similar to that of the conventionally selected aptamers. The binding assay of TATA DNA to TBP was performed in the sol-gel microfluidic chip. In this experiment, only TBP was immobilized in sol-gels during gelation. 200 pmoles of TATA DNA in a 25 μl reaction volume were introduced to the microfluidic chambers. The measured melting point of TATA DNA was 72° C. After 2 hours of incubation, the whole microfluidic channel and chambers were extensively washed with the wash buffer, as used earlier, at 15 μl/min for 30 minutes. The fluorescence intensity of the sol-gel except for the negative sol-gel was detected under the fluorescent microscope (FIG. 6). This indicates that TATA DNA indeed bound to the immobilized proteins in the sol-gel. As in FIGS. 5A-D, the intensity of the sol-gels was exponentially reduced as time passed. Therefore, it is believed that the bound TATA DNA was released from the target protein, TBP, while the electric power was being delivered. Because the obtained data also fit the equation shown in Example 2, the half-life time of the intensity decay can be extracted at the power of 450 mW, 6.4±1.55 sec, which is an acceptable value although a different constitution (TATA DNA-Cy3+TBP) compared with the previous experiment was used. This result indicates that the aptamers can be entrapped with the target protein in the sol-gel networks located in the microfluidic device, and then released freely when the ambient temperature exceeds the melting temperature of the aptamers. Moreover, the entrapment and the release of aptamers can be controlled precisely by using the microfluidic device.

### Example 4

### Verification of the RNA Aptamers from the Selective Elution

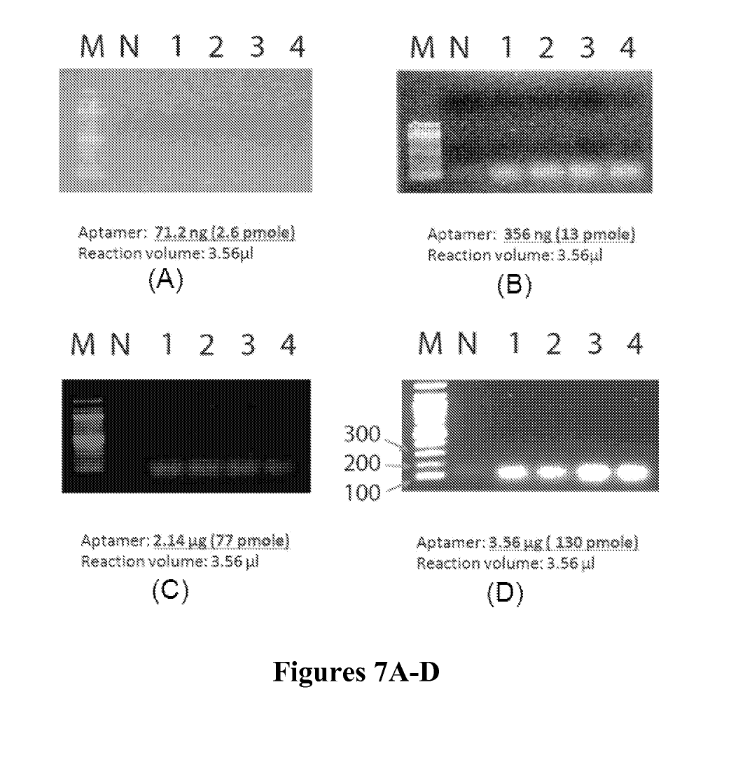

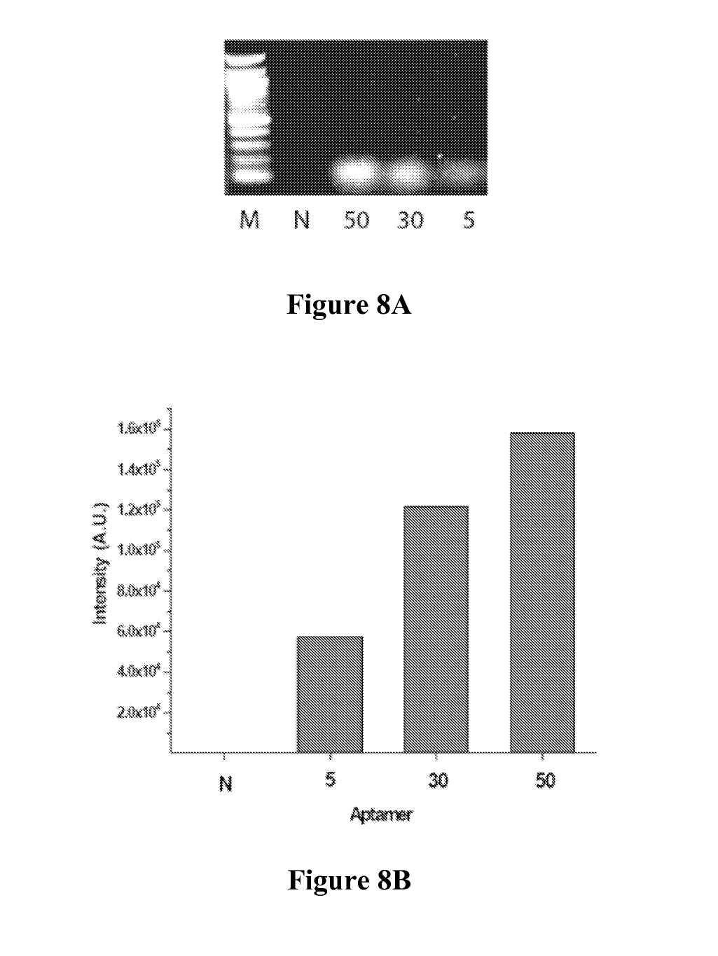

Based on the results of Examples 2 and 3, it was expected that RNA aptamers that bind to the immobilized proteins would be eluted when enough power was delivered to heat the sol-gel matrix over the embedded biomolecule's denaturing temperature. To verify that the RNA aptamers bind to the target protein rather than non-specifically to the sol-gel matrix, 4 sol-gels with immobilized TBP and a blank sol-gel for the negative control experiment were dotted in the microfluidic device. The class 1 RNA aptamer which interfered with TBP's binding to TATA DNA was selected as a reaction sample, because of its high affinity to TPB. In FIGS. 7A-D, the electropherogram substantiates that 1) the bound aptamers were successfully released from the sol-gel. The standard ladder DNA marker indicates that the bands from each sol-gel correspond to the size of the RNA aptamer's band (<100 bp); 2) since no or weak band signals were detected from the blank sol-gels (negative control), the RNA aptamers were majorly bound not to the sol-gel matrix but instead to TBP; 3) the limitation of the concentration to resolve the aptamers in the given PCR cycle is around 2.6 pmole to 13 pmole. FIGS. 8A-B compare the band intensity from each sample in the same agarose gel. The band intensities are proportional to the injected RNA aptamers. This is strong evidence that RNA aptamers bind to the target protein in the sol-gel networks.

### Example 5

### SELEX Cycle Efficiency Test

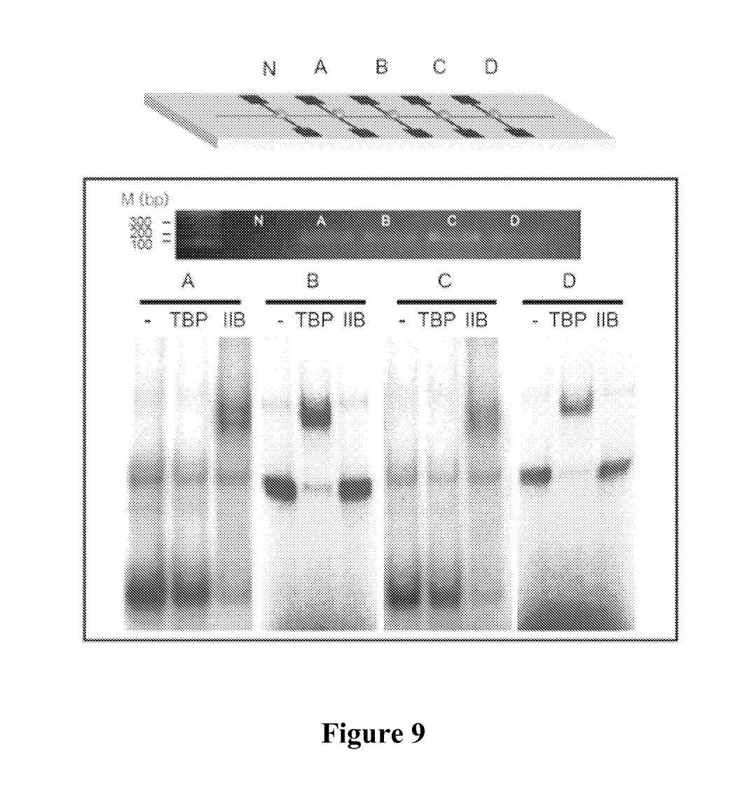

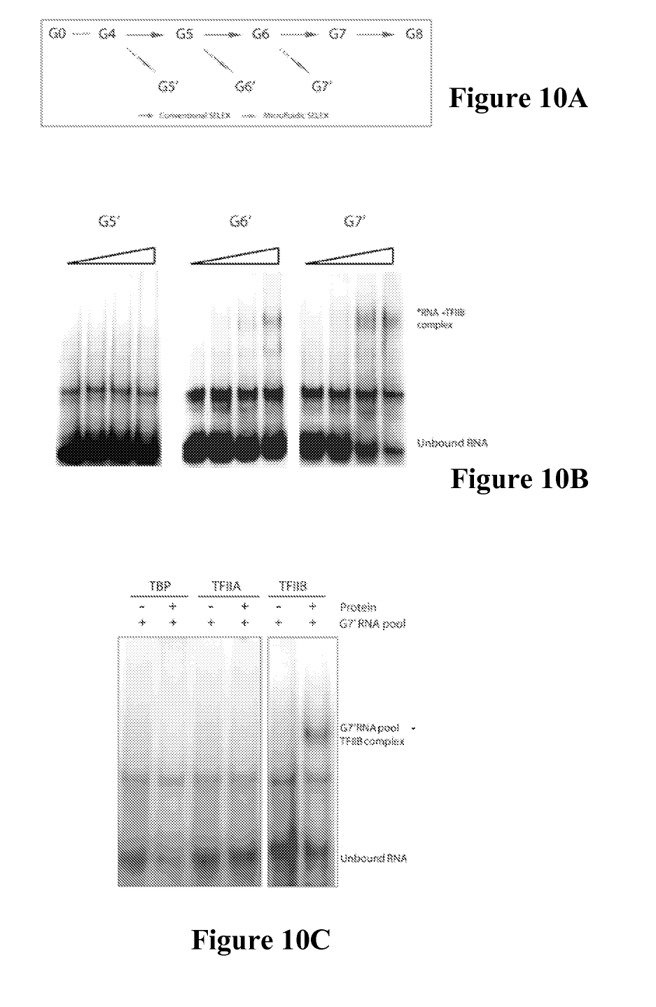

To investigate the cycle efficiency of the microfluidic SELEX chip in selection, its ability to select the aptamer from the RNA pools in different stages was tested. Prior experience demonstrated that major binding affinity between TFIIB and selected aptamer pool was first shown at the 8th round of SELEX (G8). For comparison, the microfluidic SELEX was started with known G4, G5 and G6 round of RNA aptamer pools for TFIIB selection. These RNA pools were developed with the starting pool (<2×10¹⁵ individuals) by the conventional SELEX filter binding assay. One cycle of an in vitro selection experiment was performed with TFIIB protein as target, which was immobilized in 4 sol-gels in the microfluidic SELEX device. After 1 hour incubation in the reaction microchamber, heat elution and transcription, products from each round (G4, G5 and G6) were named G5′, G6′and G7′. The affinity of these products to TFIIB was tested using EMSA. The results are depicted in FIGS. 9-10. As shown, in G6′ and G7′ but not in G5′, there is affinity between the RNA pools and TFIIB (FIG. 10B). G7′ RNA pools showed higher affinity than that of G6′. Therefore, the microfluidic SELEX chip appears to have better selection efficiency (2 cycles earlier) than that of the conventional filter binding assay. The indication that the product indeed bound to the TFIIB, and not to others, came from an EMSA with 3 different proteins (FIG. 10C). TFIIA and TBP were selected because TFIIB is a component of the polymerase II transcription machinery and forms a quaternary complex with DNA, TBP and TFIIA. Although these three proteins are very closely related to each other, G7′ product only shows affinity to TFIIB. This means the one cycle microfluidic SELEX product binds specifically to TFIIB.

### Example 6

### In vitro Selection of RNA Aptamers Against Multiple Target Proteins on Microfluidic SELEX-on-a-Chip Device

The schematic diagram for the overall experimental setup is shown in FIG. 2. Four independent experiments (four target proteins) were performed with four different aptamer concentrations. Five sol-gel droplets were spotted evenly along the microfluidic channels as described in Example 1. Each sol-gel droplet can entrap approximately 30 fmoles protein, so that a total of 120 fmoles (for four proteins) was immobilized in one microfluidic device.

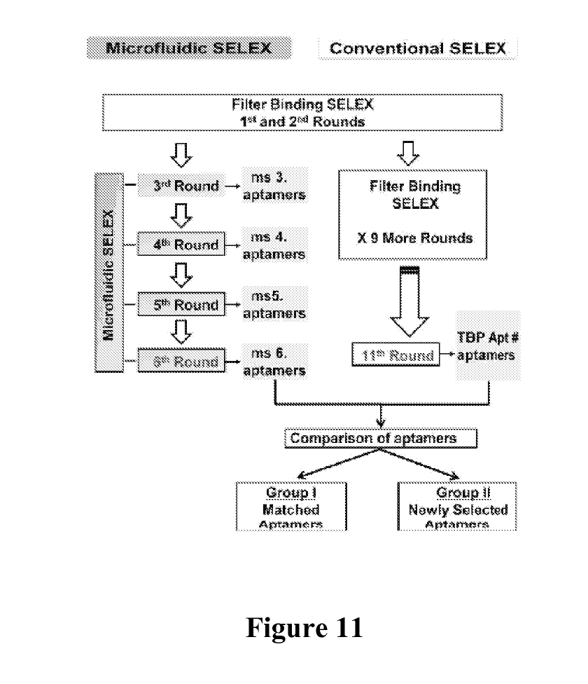

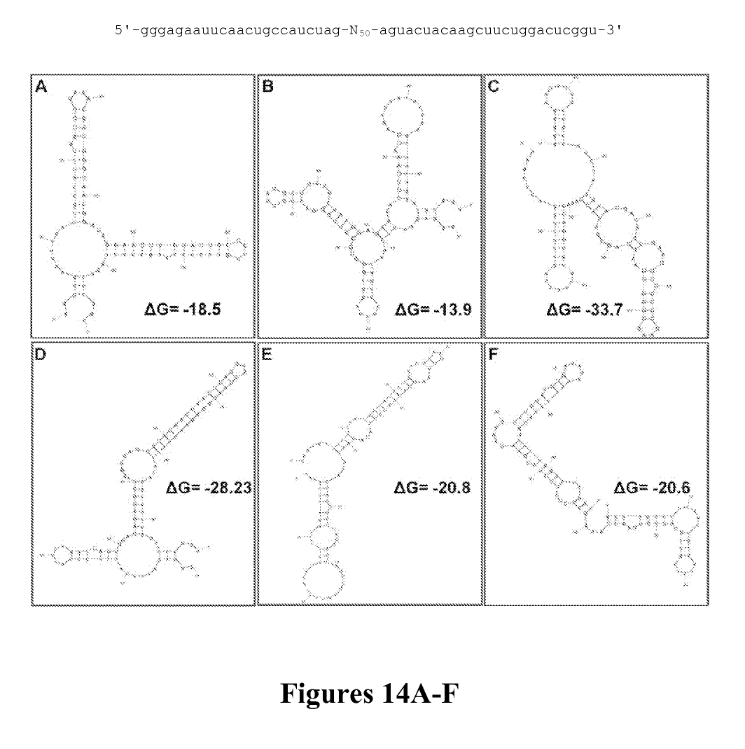

The starting pool contained ˜10¹⁵different RNA molecules. The structure of the pool member included a central 50 by long randomized region flanked by two constant regions that contain a 5′-T7 promoter to facilitate amplification by PCR (see FIGS. 14A-F). The first two cycles of selection and amplification were performed using the conventional nitrocellulose filter binding assay as previously described (Yokomori et al., Genes & Dev 8:2313-2323 (1994); Fan et al., Proc. Nat'l. Acad. Sci. USA 101:6934-6939 (2004); Sevilimedu et al., Nucleic Acids Res 36:3118-3127 (2008), which are hereby incorporated by reference in their entirety). Each RNA-protein mixture was incubated in 1× binding buffer (12 mM HEPES pH 7.9, 150-200 mM NaCl, 1-10 mM MgCl₂, 1 mM DTT), partitioned using a nitrocellulose filter, and the bound RNA was recovered by extraction with phenol and amplified to yield an enriched pool for the next cycle. This is illustrated in FIG. 11.

After the second cycle, four cycles of in vitro selection and amplification were performed using the microfluidic SELEX platform of Example 1 (FIGS. 3A, 11). Before injection of the reaction sample into the microfluidic device, the microchannel and reaction chambers were wetted with the binding buffer and blocked for 1 hour to prevent possible non-specific binding of the aptamers to either the sol-gel or the microfluidic device. Then, the reaction sample with a volume of 25 μl was injected into the device and incubated for 2 hours at the room temperature. Around 1.2 pmole of RNA species, in a 3.46 μl reaction volume, were introduced to the microfluidic chambers.

In these chips, all reactions and washing procedures were performed using a syringe pump. After incubation and washing, the sol-gel droplets in the microfluidic chambers, with a 90 μl/min flow of a binding buffer, were heated by applying currents to the electrodes using the Keithley 2400 source meter (Cleveland, Ohio). Optimal electric powers (1.5V, 450 mW) were applied to the aluminum electrodes for 2 minutes for heat elution, starting from hHSF1 droplet (chamber 4, closest to outlet) to TBP droplet (chamber 1) and negative control (chamber N, closest to inlet). The relative position of the chambers containing these sol-gel spots is shown in FIG. 3A. Carrying out heating in this order avoided undesired heat effect in subsequent chambers.

Each eluted RNA aptamer was retrieved, reverse-transcribed to cDNA, amplified, and then transcribed to RNA aptamer (FIG. 3A) as in conventional SELEX. The reverse transcription reactions were carried out using a reverse transcription kit (Invitrogen, CA). The cDNA was directly transferred to PCR step (15 cycles). The sequence of the forward and reverse primers were:

Forward
(SEQ ID NO: 3)
5′-GTAATACGACTCACTATAGGGAGAATTCAACTGCCATCTA-3′
Reverse
(SEQ ID NO: 4)
5′-ACCGAGTCCAGAAGCTTGTAGT-3′.

The PCR product's band size (˜100 bp) was analyzed by 8 M Urea polyacrylamide gel electrophoresis. Each PCR product was purified using QIAquick PCR Purification Kit (Qiagen, Germany) and then converted into RNA aptamers using a MEGAshortscript kit (Ambion, USA). Equimolar RNA aptamers against TBP, TFIIA, TFIIB, and hHSF1 were introduced into microfluidic chip for the following selection step (FIG. 3A).

The preceding examples demonstrated that aptamers specifically bind their respective protein targets and can be selectively eluted by micro-heating. Based on this SELEX-on-a-chip strategy, first protein SELEX was performed using yeast TBP which was previously selected for aptamers using conventional filter binding SELEX. As shown in FIG. 3A, TBP proteins were immobilized along with three more proteins (TFIIA, TFIIB, and hHSF1) and one negative control (without proteins) to obtain highly specific aptamers without the negative SELEX step. This also reduces the number of cycles compared to conventional SELEX (Jenison et al., Science (New York, N.Y.) 263:1425-1429 (1994), which is hereby incorporated by reference in its entirety).

To obtain high affinity and specific aptamers, in case of conventional macro-scale SELEX, the full set of random aptamer pool was added (around 10¹⁵˜1.7 nM). This uses more amounts of pool than those of proteins since competition will increase the selectivity of aptamers among pool. Therefore, the microfluidic device for SELEX should be able to hold the target proteins at least 1.7 pM (1000 times less than pool). However, the SELEX microfluidic device can only hold 30 fmol (0.6 ng) of TBP proteins in each 7 nl of sol-gel droplet and, thus, total 120 fmol of protein can be immobilized as shown in FIG. 3B. In case of microfluidic device or chip-based miniaturized assay, the small amounts of target proteins (around 14 fold less) immobilized can be more problematic since it loses the complexity of pool. Therefore, during the initial rounds of microfluidic SELEX, the filter binding SELEX was used, and then after getting the enriched pool of aptamers, microfluidic SELEX was started to obtain specific and full variety of aptamers. It should be appreciated that using larger multiplexed devices will allow for direct screening of a random nucleic acid pool without the need to first perform conventional SELEX.

As shown FIG. 11, TBP aptamer selection was performed using microfluidic SELEX and the results were compared with conventional filter binding SELEX (Fan et al., Proc. Nat'l. Acad. Sci. USA 101:6934-6939 (2004), which is hereby incorporated by reference in its entirety). After two initial filter binding SELEX, four consecutive rounds of microfluidic SELEX were performed. In the case of conventional SELEX of yeast TBP, TBP aptamers can be obtained after 11 cycles of SELEX, with several additional negative selection cycles. In these examples, highly specific and strong affinity aptamers were obtained after only 3 cycles of microfluidic SELEX, even without negative SELEX. The resulting aptamer populations compare with those reported in Example 5 above. Since TBP target protein was immobilized at the first position with other sol-gel droplets containing TFIIA (position 2), TFIIB (position 3) and hHSF1 (position 4) as competitors and with no protein droplet (N), there is no need for additional negative SELEX step. This further reduces the cycles of microfluidic SELEX and increases the specificity of aptamers selected (FIG. 3A).

Using the final selected pool from 6^{th} round (ms-6), 38 individual aptamers were obtained and sequenced. These individuals belong to 20 clones and the sequences of clones are listed in Table 5 below. Based on the above-noted comparison between sequences of aptamers isolated from microfluidic SELEX with those previously selected by the conventional filter binding (Fan et al., Proc. Nat'l. Acad. Sci. 101:6934-6939 (2004), which is hereby incorporated by reference in its entirety) using sequence alignment, and it was found that they had 100% homology (aptTBP-#17/ms-6.16 and aptTBP-#1/ms-6.38) and 98% homology (aptTBP#13/ms-6.4) as listed in group I and newly isolated aptamer sequences listed in group II, respectively. Except for ms-6.7, there is no shared consensus sequence among these clones. In the case of ms6-#4, the most abundant sequence (8 of 38) of the microfluidic SELEX, it was a high affinity aptamer (with one base-pair mismatch) isolated from the previous study (Fan et al., Proc. Nat'l. Acad. Sci. USA 101:6934-6939 (2004), which is hereby incorporated by reference in its entirety). These results show that the successful isolation of aptamers can be achieved using the microfluidic SELEX.

### Example 7

### Protein-Aptamer Binding Assay Using Sol-gel Based Array Chip

The enriching step of TBP aptamers in microfluidic SELEX experiment were further studied. Each round of aptamer pools (ms-3, ms-4, and ms-5) against TBP were collected, and then cloned and sequenced. The sequences are shown in Table 1-4 below. Surprisingly, aptamers (TBP apt#1) can be selected even after 3 cycles (first round of microfludic SELEX). In addition, three aptamer classes observed in first cycle of microfluidic SELEX-on-a chip, ms-3 (ms-3.1, ms-3.2, and ms-3.25), were shared over 60% (31-nt of 50). In the case of clone ms-3.1, it was isolated 4 times in 23 individuals. Moreover, clone ms-3.3 was fully overlapped with ms-4.20, ms-5.4, ms-6.38 and aptTBP-#1. A seven nucleotide stretch shared by ms-3.15 and ms-3.23 were highly conserved and widely observed in sequence data. Therefore, using microfludic SELEX, a high affinity aptamer can be obtained even after first cycle of microfludic SELEX.

To further investigate the binding activity of the newly isolated aptamers from microfluidic SELEX, aptamers were labeled with Cy-3 individually. The selected individual aptamers against TBP were first transcribed using a MEGAshortscript kit (Ambion, USA). Briefly, after PCR amplifying the aptamer construct DNAs, 1 μg of the amplified templates was used for in vitro transcription according to the manufacturer's protocol. Thereafter, aptamers were labeled with Cy3-dUTP using terminal deoxynucleotidyl transferase (TdT). RNA aptamer (1 nmol) was incubated for 4 hours at 37° C. with 2 nmol Cy3-dUTP (E-biogen, Korea), 20 units of TdT (Fermentas) in 200 mM potassium cacodylate, 25 mM Tris/HCl (pH 6.6), 0.25 mg/ml bovine serum albumin, 5 mM CoCl₂ and 0.5 mM deoxynucleotide triphosphate in a final volume of 20 μl. 10 unit RNase inhibitor (Boehringer Mannheim) was added. The reaction was stopped by addition of EDTA. The labeled RNA was extracted by phenol/chloroform/isoamylalchol treatment and recovered by ethanol precipitation in the presence of 0.3 M sodium acetate.

Binding of RNA pools to TBP was tested using sol-gel chip assay. Within 8 diameter wells of 96-well type plates (SPL, Korea), six duplicate spots were printed along with negative controls (no protein) and positive controls (Cy-3 labeled proteins). Sol-gel protein chip printing methods were used as described previously (Kim et al., Anal Chem 78:7392-7396 (2006), which is hereby incorporated by reference in its entirety). The wells were soaked with 100 μl of PBS solution and incubated for 2 hr with blocking buffer (binding buffer containing 20 μg/ml tRNA). After washing, aptamers labeled with Cy-3 (labeled by TdT enzyme) were incubated for 2 hr in each well and then washed 3 times for 15 min. The resulting plate chip well was scanned and analyzed using a 96-well fluorescence scanner and the appropriate software program (FLA-5100 and Multi-gauge, Fuji Japan). The background intensity was subtracted from the signal intensity of each spot (LAU/mm²).

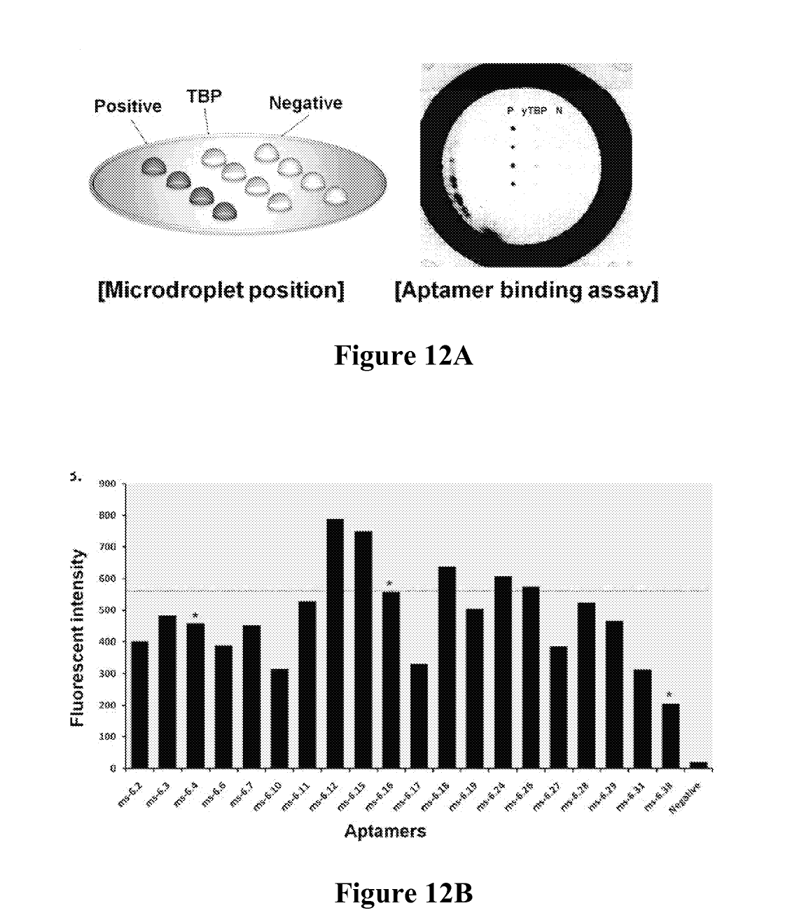

Individual binding activity was calculated by fluorescent intensity of sol-gel microdroplets (FIG. 12A). The results are shown in FIG. 12B. Some of ms-6 aptamers specifically bind TBP proteins better than those previously selected by the conventional filter binding (FIG. 12B). Interestingly, aptamer ms-6.16 showed higher binding activity than aptamer ms-6.4. This result is reasonable when compared to the dissociation constant (k_{d}) between TBPap-t#17 and TBPapt-#13 (Fan et al., Proc. Nat'l. Acad. Sci. USA 101:6934-6939 (2004), which is hereby incorporated by reference in its entirety). Together, these results confirm that the microfluidic device can enrich aptamers even after the first round of selection.

TABLE 1
TBP Aptamers Selected By Microfluidic SELEX, Round 3

| | | Fre- | Sequence |
|---|---|---|---|
| Identifier | Sequence | quency | ID No: |
| ms 3.1 | UCCCGGCCGCCAUGGCGGCCGCGGGAAUUCGAUAUCACUAGUGAAUUCGC | 4 | 5 |
| ms 3.2 | UCCCGGCCGCCAUGGCGGCCGCGGGAAUUCGAUUAUCCACAGAAUCAGGG | 1 | 6 |
| ms 3.25 | CCGGCCGCCAUGGCGGCCGCGGGAAUUCGAUCAAAAGGCCAGGAACCGUA | 1 | 7 |
| ms 3.9 | CACCCUAAUCAGAGCUGCUAGUUAGGGCGUACAAAACUGCACUUCUAUC | 2 | 8 |
| ms 3.15 | CCAGGAGC | 1 | 9 |
| ms 3.23 | CCUAUGCCAGUGAAUCUCCGCGAGCUUUAAUGACAGGAGCUCCUCAGUU | 1 | 10 |
| ms 3.3 | AGAUCACGAAAAAGCGGAAUUGAGGUACCCAAGAGCUAAAAAAAAGACAUCC | 1 | 11 |
| ms 3.4 | UUCUCGCGAAGACCUUGAGCAACUUGCAACCUCCAGAGCAUGACAAAUGG | 1 | 12 |
| ms 3.5 | GGAGCAAACACCAACGCCUGAUCGCUCGACCGACACAACCAAAUAAAAAG | 1 | 13 |
| ms 3.8 | CCCGCAGCAUGGUGGCGCGUCGGUGAUACGUGAGACUGGGUGAAAGCCAG | 1 | 14 |
| ms 3.13 | UUACGUGCAUGAAAACCCAACACGUGGCGCAAAACUAACACACAGGGAGU | 1 | 15 |
| ms 3.14 | GGAAGCUGAAGGGCACGAAAGGCUGUUGAGCUGUUAGAUCCGACUUGCAG | 1 | 16 |
| ms 3.16 | UCGAGAACCAUCCUACCAGACUGGGAAGUGCAGGAGGGAAGAUGACCGGA | 1 | 17 |
| ms 3.17 | AAAGAGCCAAAGGCGCACAUGCCGGUUCAGAAAAAAAAAACACCAGAAACUC | 1 | 18 |
| ms 3.18 | AUACCCAAGGGGCCACCAAGGGAGAGUUCAGGGUGGGCGAAUUACGUACU | 1 | 19 |
| ms 3.19 | UCGUAAAUCAAAAAAAGGAGGGAGGGUUACAAAGGGACGAACAGAACAGG | 1 | 20 |
| ms 3.21 | UAGAGGGAGGGUAGUAUCCAUGGAAUCUGAACGAACAUCAAAACAUGAAU | 1 | 21 |
| ms 3.22 | GACAGCACAAACGAUAAUCACUGGAACAAACUCGGCCUUGCGUUGGAAGU | 1 | 22 |
| ms 3.26 | UGACCUAAGAUCAGGUUAGGAGUUUUUUAACUAAGGUGAGUGACGAAGCC | 1 | 23 |

TABLE 2
TBP Aptamers Selected By Microfluidic SELEX, Round 4

| | | Fre- | Sequence |
|---|---|---|---|
| Identifier | Sequence | quency | ID No: |
| ms 4.20 | AGAUCACGAAAAAGCGGAAUUGAGGUACCCAAGAGCUAAAAAAAGACAUCC | 3 | 24 |
| ms 4.25 | CACGGGCAAGACAAGACAAAUACUGUCAGUCGACCAUGAGCCUGACCGCC | 3 | 25 |
| ms 4.1 | CCACUAACCAUGCGGAAAAAGACCACAGCCAACAUAAAAACGAACCAGCA | 1 | 26 |
| ms 4.18 | CAAUAAAUCGAAGUCCACACGGCAAUCCAGAAAAACGACACAGAAGCGGU | 1 | 27 |
| ms 4.21 | CAGAGGCAAGCGAAGACCCGCGUGCACAAAACCGACAGACCAGGAAUUGG | 1 | 28 |
| ms 4.7 | CGAGACGAUAAGGGCGAGGGUCAGUAAAGGGCAGGGAUGCAACAAACAGA | 1 | 29 |
| ms 4.23 | GCCAAGGGAAAGGGCAAGAAAGGGUCGGGGAAUUCCCACGCAGAUCUAGG | 1 | 30 |
| ms 4.6 | CCGCCAAAGUAAAGAAAGGAGGAGGAGGAACGCGGGCACACCGAGCAACA | 1 | 31 |
| ms 4.8 | AGGAGCACGG | 1 | 32 |
| ms 4.2 | CGAACGUCCGGUAGCAUGAACGAAUAGGGCUUGGGUGGGCAAAGAGGGAG | 1 | 33 |
| ms 4.5 | CAAGGGAGAGGAAGAUCAGAAAGGGAAAGGGAACACUGGGACACGUUGAG | 1 | 34 |
| ms 4.11 | CCCUAUCCGGAUGAUCUCAGUUCACUGUUAAAUUCUCUGGAAUUGACCGU | 1 | 35 |
| ms 4.12 | CGGAAUCGAGAGCCAAGUGUGAUGGGAGGGAAUAUCUUGAGGGAAACGGG | 1 | 36 |
| ms 4.16 | GCCGAGCAGUAAACCUGACAACAUGGGUUGGGAAGGGUAGGGCCGUGAGU | 1 | 37 |
| ms 4.17 | ACGCUUGAGUAGGCUAGUUGUUACUUUGUUCAGGUUCGCGAAGAACACCA | 1 | 38 |
| ms 4.22 | CCGACUGAUGUAGAAUOUGGCCAUUCGCCACAAAGGAUGAAGCCUAGUGG | 1 | 39 |
| ms 4.28 | UGGGCUGGGUCUCGCGAAAUUUCAAUCCGAAUAAGAUAAACCAAGCCUUG | 1 | 40 |
| ms 4.30 | GCGCGGGAUGGGAGCGAACACGAGCGACACCGAAGAAAGCGAAGCAAACC | 1 | 41 |
| ms 4.34 | UAAGGCGACCCAGGAACCAGAGUCCGCCCCUUGAUCGAGAAAGACACUUG | 1 | 42 |
| ms 4.36 | CGGAGGAGGGCGGGGUUGGUGGAUGUAUCGUUGAAAUUCCUCCACAGACG | 1 | 43 |
| ms 4.48 | GGCCGCGGGAAUUCGAUUUAGGGAGAAUUCAACUGCCAUCUAGCCAGGAG | 1 | 44 |

TABLE 3
TBP Aptamers Selected By Microfluidic SELEX, Round 5

| | | Fre- | Sequence |
|---|---|---|---|
| Identifier | Sequence | quency | ID No: |
| ms 5.5 | GGCCGCGGGAAUUCGAUUGAGAAUUCAACUGCCAUCUAGCCAGGAGCACG | 1 | 45 |
| ms 5.7 | AUCUAGCCAGGAGCACG | 1 | 46 |
| ms 5.13 | CCAGGAGCACGG | 3 | 47 |
| ms 5.10 | CAUGGGCAAGACAAGACAAAUACUGUCAGUCGACCAUGAGCCUGACCGCC | 2 | 48 |
| ms 5.9 | UCCCGGCCGCCAUGGCGGCCGCGGGAAUUCGAUUACCGAGUCCAGAAGCU | 1 | 49 |
| ms 5.21 | UCCCGGCCGCCAUGGCGGCCGCGGGAAUUCGAUUACCGAGUCCAGAAGCUU | 1 | 50 |
| ms 5.17 | UCCCGGCCGCCAUGGCGGCCGCGGGAAUUCGAUUACUCACUAUAGGGAGAA | 1 | 51 |
| | UUCAACUG | | |
| ms 5.1 | CGCUAGAAACUACAAACGGGGUUGGGUGGAAACGGAUGAGGGAAACUUAG | 1 | 52 |
| ms 5.4 | AGAUCACGAAAAAGCGGAAUUGAGUUACCCAAGAGCUAAAAAAAGACAUCC | 1 | 53 |
| ms 5.8 | CAGAGCACCCGAUAGCUGUGUGGUUGGUAUUUACGCCUACUAGCUCGCAG | 1 | 54 |
| ms 5.12 | CGAAGCCCACACGACC | 1 | 55 |
| ms 5.18 | CCAUACAUGGGCAACGAUGCUACUCCAAGACGCAUGACCC | 1 | 56 |
| ms 5.20 | AGAUACCCCGAUGAUGCGCAGCCCAGUCCUCGCUGCCGCCAG | 1 | 57 |
| ms 5.22 | GUCGCGUGUUUGCGUAUACUCUGACCUGAAAUGCGAAUAUCGCUUACGAG | 1 | 58 |
| ms 5.24 | UAAAAACGGGACCCACUCCACCCGUCUAGGAGGGAUAUCCCGAAAACACG | 1 | 59 |
| ms 5.25 | GGGGGGGCCUGGGUAAGAUAAGCUGGCCUGUGCUCGGUGGGCUUGUUAUC | 1 | 60 |
| ms 5.26 | GAUAUGGGGGGACAAUCCCACCGGUGAAGACGUGUUCAAUUAAAGGAACG | 1 | 61 |

TABLE 4
TBP Aptamers Selected By Microfluidic SELEX, Round 6

| | | Fre- | Sequence |
|---|---|---|---|
| Identifier | Sequence | quency | ID No: |
| ms 6.7 | CAGGAGCACGG | 12 | 62 |
| ms 6.4 | CAUGGGCAAGACAAGACAAAUACUGUCAGUCGACCAUGAGCCUGACCGCC | 8 | 48 |
| ms 6.2 | UGUUGUUAAAUCUUGCUGGACCGUCCCCAUGCUUACGCCCGUCGUUC | 1 | 63 |
| ms 6.3 | CCAUGACGCAAAAUUGGAGGCAUAUGGAACGGAAACUCCGGGAAAGUAGA | 1 | 64 |
| ms 6.6 | UUUGUAUACUUUUUCGCUUGUGUCGUUGAACGUAAGUACUCUGUCUGCAU | 1 | 65 |
| ms 6.10 | CGGAUCAUGCCCUCAGGCAGUUUCGCCGAACCGAUAAAACUUUUGCUUGU | 1 | 66 |
| ms 6.11 | UGCUAUGUAGAGUGAUUGCUGAGGUGGGUUUUUUGUGUUAGGGAAGGGAGAUUGU | 1 | 67 |
| ms 6.12 | UGGUAAACCACGGGUAACGGAUAGGAAGUUGUAUUGCCCU | 1 | 68 |
| ms 6.15 | GGGUGCCUUGGGAAUCUUAUGAUCCAGCUAAGGAGAACACUUGAAAGCAA | 1 | 69 |
| ms 6.16 | ACGACACCGAAGGCGCCCCGAAGGGGGGCAAGGAGCCAUACCAAACCAGG | 1 | 70 |
| ms 6.17 | GGGAGGCGGGCGAGUUUCGGGACUGGCACCCUCAAUCCCAUCAAACCAGA | 1 | 71 |
| ms 6.18 | CAGUGGACAGAGGCUCGGGAGGGUACAACUAACUUAGGGACUAAGGGAGA | 1 | 72 |
| ms 6.19 | GUGUCCUUGGCUUGCGUAUGCUUAUCUGCUAACGUCCAAGGUUGUUUAUG | 1 | 73 |
| ms 6.24 | GACAAGGUAAUUAGACGGCAAGAGAAUAAACGAGGUCCCACCAGCAUCGC | 1 | 74 |
| ms 6.26 | GCAUUCUUACCCAAAGCCCUCGUCUACGAAUAAUCUUUGUAUGUGAUA | 1 | 75 |
| ms 6.27 | CCGAGGCGCACCUAGCAGCGUUGAGUAGGACCGAGAAACAUAAGUAUGAA | 1 | 76 |
| ms 6.28 | CAAUCGAGGGACGGGCCAGACGGGAAAGGGGAUUGUCUUACACAGAGGCC | 1 | 77 |
| ms 6.29 | GCGGACCCGCCGAAAACGCAACCGUGCACAAUUCUGAGCAUGGGCGGGCC | 1 | 78 |
| ms 6.31 | CGCCCAGGUGGCGAAGCGGAGACUGAAUCUAUGUCACCUUAUCUUGGCA | 1 | 79 |
| ms 6.38 | AGAUCACGAAAAAGCGGAAUUGAGUUACCCAAGAGCUAAAAAAAAGACAUCC | 1 | 80 |

TABLE 5
Group I and Group II TBP Aptamers Selected By Microfluidic SELEX

| | | | Fre- | Sequence |
|---|---|---|---|---|
| Group | Identifier | Sequence | quency | ID No. |
| | TBPApt #1 | AGAUCACGAAAAAGCGGAAUUGAGGUACCCAAGAGCUAAAAAAAGACAUCC | — | 24 |
| | ms 3.3 | AGAUCACGAAAAAGCGGAAUUGAGGUACCCAAGAGCUAAAAAAAAGACAUCC | 1 | 11 |
| | ms 4.20 | AGAUCACGAAAAAGCGGAAUUGAGGUACCCAAGAGCUAAAAAAAGACAUCC | 3 | 24 |
| | ms 5.4 | AGAUCACGAAAAAGCGGAAUUGAGUUACCCAAGAGCUAAAAAAAGACAUCC | 1 | 53 |
| | ms 6.38 | AGAUCACGAAAAAGCGGAAUUGAGUUACCCAAGAGCUAAAAAAAAGACAUCC | 1 | 80 |
| Group I | TBPApt #13 | CAUGGGCAAGACAAGACAAAUACUGUCAGUCGUCCAUGAGCCUGACCGCC | — | 81 |
| | ms 4.25 | CACGGGCAAGACAAGACAAAUACUGUCAGUCGACCAUGAGCCUGACCGCC | 3 | 25 |
| | ms 5.10 | CAUGGGCAAGACAAGACAAAUACUGUCAGUCGACCAUGAGCCUGACCGCC | 2 | 48 |
| | ms 6.4 | CAUGGGCAAGACAAGACAAAUACUGUCAGUCGACCAUGAGCCUGACCGCC | 8 | 48 |
| | TBPApt #17 | ACGACACCGAAGGCGCCCCGAAGGGGGGCAAGGAGCCAUACCAAACCAGG | — | 70 |
| | ms 6.16 | ACGACACCGAAGGCGCCCCGAAGGGGGGCAAGGAGCCAUACCAAACCAGG | 1 | 70 |
| Group II | ms 6.7 | CAGGAGCACGG | 12 | 62 |
| | ms 6.2 | UGUUGUUAAAUCUUGCUGGACCGUCCCCAUGCUUACGCCCGUCGUUC | 1 | 63 |
| | ms 6.3 | CCAUGACGCAAAAUUGGAGGCAUAUGGAACGGAAACUCCGGGAAAGUAGA | 1 | 64 |
| | ms 6.6 | UUUGUAUACUUUUUCGCOUGUGUCGUUGAACGUAAGUACUCUGUCUGCAU | 1 | 65 |
| | ms 6.10 | CGGAUCAUGCCCUCAGGCAGUUUCGCCGAACCGAUAAAACUUOUGCUUGU | 1 | 66 |
| | ms 6.11 | UGCUAUGUAGAGUGAUUGCUGAGGUGGGUUUUUUGUGUUAGGGAAGGGAGAUUGU | 1 | 67 |
| | ms 6.12 | UGGUAAACCACGGGUAACGGAUAGGAAGUUGUAUUGCCCU | 1 | 68 |
| | ms 6.15 | GGGUGCCUUGGGAAUCUUAUGAUCCAGCUAAGGAGAACACUUGAAAGCAA | 1 | 69 |
| | ms 6.17 | GGGAGGCGGGCGAGUUUCGGGACUGGCACCCUCAAUCCCAUCAAACCAGA | 1 | 71 |
| | ms 6.18 | CAGUGGACAGAGGCUCGGGAGGGUACAACUAACUUAGGGACUAAGGGAGA | 1 | 72 |
| | ms 6.19 | GUGUCCUUGGCUUGCGUAUGCUUAUCUGCUAACGUCCAAGGUUGUUUAUG | 1 | 73 |
| | ms 6.24 | GACAAGGUAAUUAGACGGCAAGAGAAUAAACGAGGUCCCACCAGCAUCGC | 1 | 74 |
| | ms 6.26 | GCAUUCUUACCCAAAGCCCUCGUCUACGAAUAAUCUUUGUAUGUGAUA | 1 | 75 |
| | ms 6.27 | CCGAGGCGCACCUAGCAGCGUUGAGUAGGACCGAGAAACAUAAGUAUGAA | 1 | 76 |
| | ms 6.28 | CAAUCGAGGGACGGGCCAGACGGGAAAGGGGAUUGUCUUACACAGAGGCC | 1 | 77 |
| | ms 6.29 | GCGGACCCGCCGAAAACGCAACCGUGCACAAUUCUGAGCAUGGGCGGGCC | 1 | 78 |
| | ms 6.31 | CGCCCAGGUGGCGAAGCGGAGACUGAAUCUAUGUCACCUUAUCUUGGCA | 1 | 79 |

### Example 8

### Binding Affinity (K_{d}) Measurement of Selected Aptamers

For binding affinity assay, five different concentrations of TBP (from 0 to 800 nM) were prepared and protein containing sol-gel mixtures were dropped on the surface of the 96-well. These sol-gels were arrayed using the non-contacting dispensing machine according to the manufacturer's protocol (sciFLEXARRAYER, Scienion). Single spot volume was around 50 nl and the selected aptamers were labeled by end labeling method. Each aptamers (200 pmoles) was incubated in 1× binding buffer (12 mM HEPES pH 7.9, 150 mM NaCl, 1 mM MgCl₂, 1mM DTT) for 1 hour at room temperature. After washing 3 times in 0.2% Tween20 treated 1× binding buffer, the resulting spots were scanned and analyzed by FLA-5100 scanner. The dissociation constants (k_{d}) were calculated by plotting the fluorescent intensity of the bound aptamers versus the TBP concentrations and then fitting the data points to non-linear regression analysis performed using Sigmaplot 10.0 software with the following equation:

y=(Bₘₐₓ·RNA aptamer)/(k_{d}+ssDNA)

where y is the degree of saturation, Bₘₐₓ is the number of maximum fluorescent activity, K_{d} is the dissociation constant.

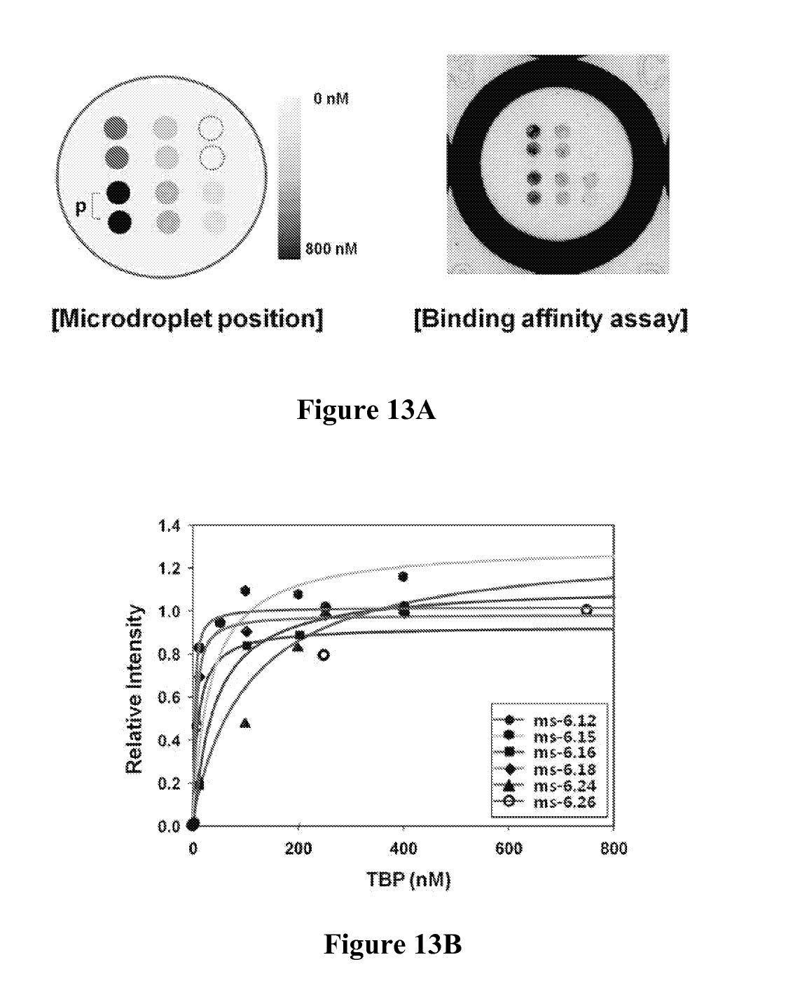

For the binding affinity calculation, the six ms-aptamers (ms-6.12, 15, 16, 18, 24, and 26) which have the highest fluorescent activity in sol-gel array were selected. The non-contacting dispensing arrayer was used as described above. The assay for K_{d} calculation in pin type arraying system has been done, but distinguishable signals were not shown in the low concentration range. This phenomenon relates to the detection volume, the number of protein species in a single spot, and the sensitivity of probing materials. As shown in FIGS. 13A-B, the 10 fold up-scaled sol-gels were well dropped without contamination between cross spots. These droplets can hold enough target protein (from 0 to 800 nM) for the binding affinity measurement of Cy-3 labeled aptamers.

The dissociation constants (K_{d}) of the all aptamers were found to be in the low nanomolar range except to ms-6.24 [K_{d} for ms-6.12, 2.7 nM; ms-6.15, 13.2 nM; ms-6.16, 8.3 nM; ms-6.18, 4.5 nM; ms-6.24, 92.53 nM; ms-6.26, 10.56 nM]. As mentioned above in the sequence comparison section, ms-6.16 corresponded to previously selected TBP aptamer #17. In the case of #17, the binding affinity was measured by EMSA and its K_{d} showed in the range from ˜3 to 10 nM. Interestingly, ms-6.16 has a K_{d} of ˜8 nM in the sol-gel chip assay described herein. Moreover, ms-6.12 showed highest affinity, with a K_{d} of 2.7 nM measured by this assay. This result has a thread of connection with binding activity test (FIG. 12B).

Secondary structure models of aptamers were predicted with the Mfold program and the most stable predicted folds are shown in FIG. 14. No apparent sequence- or secondary structure-similarity among six aptamers was observed.

### Example 9

### Identification of Selected TFIIA-, TFIIB-, and hHSF1-Specific Aptamers

The tetra-plex selection procedure of Example 6 also afforded aptamer populations specific for TFIIA, TFIIB, and hHSF1. The aptamer populations of the 6^{th} round selection were sequenced, and are identified in Tables 6-8 below.

TABLE 6
TFIIA Aptamers Selected By Microfluidic SELEX, Round 6

| | | Fre- | Sequence |
|---|---|---|---|
| Identifier | Sequence | quency | ID No: |
| TFIIA ms 6-2 | AGGAGCACG | 5 | 83 |
| TFIIA ms 6-12 | CATGGGCAAGACAAGACAAATACTGTCAGTCGACCATGAGCCTGACCGCC | 4 | 84 |
| TFIIA ms 6-3 | TCCCGGGGCATGGCGGCCGCGGGAATTCGATTACCGAGTCCAGAAGCTTGT | 1 | 85 |
| TFIIA ms 6-6 | AAAAAGGGATTCCCTACGGGACTAATAGGGAGGGAATAGTGACCTTAACA | 1 | 86 |
| TFIIA ms 6-7 | CATGGGCAAGACAAGACAAATACTGTCAGTCGACCACGAGCCTGACCGCC | 1 | 87 |
| TFIIA ms 6-8 | CCCGCAAGAATTGCTCCACCCTCTCAACCCCTACGACCC | 1 | 88 |
| TFIIA ms 6-9 | GAACAAGGGGGGGCTCGCAAAAAGGGCAGGGATTAGTTGAAAAAAACCAG | 1 | 89 |
| TFIIA ms 6-11 | CCGGCCGCCATGGCGGCCGCGGGAATTCGATTACCGATCCAGAAGCTTGT | 1 | 90 |
| TFIIA ms 6-13 | GGGAGAATTCAACTGCCATCTAGGCAGTTGAATTCTCCCTATAGTGAGTC | 1 | 91 |
| TFIIA ms 6-14 | TCCCGGCCGCCATGGCGGCCGCGGGAATTCGATTACCGAGTCCAGAAG | 1 | 92 |
| | CTTGT | | |
| TFIIA ms 6-16 | CTCTGCATTTTCCTCGGCACCTTGGACACCCGTATTAACG | 1 | 93 |
| TFIIA ms 6-20 | CCACGTTGCGTGTTGGACGGACTTGCTGAAATCTTAATCCACCACCCACG | 1 | 94 |
| TFIIA ms 6-23 | CGGGCCAAAGGAACCGAGCAGAAGCGCCGCGTTCAAGGCAACCACCAGA | 1 | 95 |
| TFIIA ms 6-24 | CGCGTCTCCACCGTGATTTGCATGGAGTTTGGCTAATATACTCCGGCCCC | 1 | 96 |
| TFIIA ms 6-25 | TTTTCTCATTCGCTTGCTGATGCCTCAAAGGCCAGGCCGAAAGCCCTAA | 1 | 97 |
| TFIIA ms 6-26 | TTGCGATACAAGACCTAAATGTCTGCGTTCTTTACCGCCG | 1 | 98 |

TABLE 7
TFIIB Aptamers Selected By Microfluidic SELEX, Round 6

| | | Fre- | Sequence |
|---|---|---|---|
| Identifier | Sequence | quency | ID No: |
| TFIIB ms 6.10 | AGGAGCACG | 9 | 99 |
| TFIIB ms 6.1 | CCGTAGGCATGTCGTAGGCCAAGTGAAGCTGTTGAAGCGCGTATCGCGGC | 1 | 100 |
| TFIIB ms 6.3 | GGAAGGCGGGAGCGGTTAGGGCTTAGGTGAATGTCGAATGACATGAGGCT | 1 | 101 |
| TFIIB ms 6.5 | CCTATTTACCCAGCGTCCTAGTTTTATTGAGTACTAGCTTTTGCTCCAAG | 1 | 102 |
| TFIIB ms 6.7 | TCGTGTCCATCCACGAACCTGGCATCCGCGACTTATTTTG | 1 | 103 |
| TFIIB ms 6.11 | ACAGAACTCTTGCCGCCCCCTCCTTAGCTGGGGACCTGAT | 1 | 104 |
| TFIIB ms 6.12 | CATGGGCAAGACAAGACAAATACTGTCAGTCGACCATGAGCCTGACCGCC | 1 | 84 |
| TFIIB ms 6.13 | GAGACGTTGATGCTCAAGCTCTGGAGACATATGATACCCCCACGAACAGG | 1 | 105 |
| TFIIB ms 6.14 | GGGGATGGAAGTTTCGACGGTACCAGAATCGGGTAGCTCCGAGAGGGCCC | 1 | 106 |
| TFIIB ms 6.18 | TGACTGTGCATCAGGCCTATGGCGCCGTGCGCCCCCGAACCAGACTAGCG | 1 | 107 |
| TFIIB ms 6.22 | CCAATTGATTGATTTCATCGCTCTCTGCGGTGGCTTAGTTTTCGACAGG | 1 | 108 |
| TFIIB ms 6.23 | GTAACAACTTAAGCCCTGATTCCGACTGCCTGCACTAA | 1 | 109 |
| TFIIB ms 6.24 | CGATCGTTTCGGTGCGGCCCGCCGGGCCTGAGCGATTGAAGCCTAGGACC | 1 | 110 |
| TFIIB ms 6.28 | ACACGCGGACTCCCAAAAGGCAACGCCTTAAAGCCCGCCC | 1 | 111 |
| TFIIB ms 6.34 | AAAGATCAAAAGTGTAAAGTTGAGTGTGCTAGCGTCACGTTGAACGGCG | 1 | 112 |

TABLE 8
hHSF Aptamers Selected By Microfluidic SELEX, Round 6

| | | Fre- | Sequence |
|---|---|---|---|
| Identifier | Sequence | quency | ID No: |
| hHSF ms 6.1 | CATGGGCAAGACAAGACAAATACTGTCAGTCGACCATGAGCCTGACCGCC | 4 | 84 |
| hHSF ms 6.2 | CCGCAGGGAGCAAAAGTTGGTTAGCCCAGAAAGCCAGAATAAAGCAATCC | 1 | 113 |
| hHSF ms 6.3 | ATGACCGAAAGGCACCGAGGCTCACCAAACGTAGCCGCCC | 1 | 114 |
| hHSF ms 6.4 | GAAGACAGGCACACATTCACGCCAAGAAAGCGCCCCGAAGAACAGCAAAA | 1 | 115 |
| hHSF ms 6.5 | AAGATTGCGGAGTGCTCAACTACTACGTTCCACGCATAGC | 1 | 116 |
| hHSF ms 6.8 | CGAGGTGGGCGGAAGGTGTGGCTAGAGGCGGTTGCATGACTCTGACCCGG | 1 | 117 |
| hHSF ms 6.9 | GCGCGATGGTAAACGAGGCTCTAAAAGAAGCATAGGCTTAGGGCATGCCA | 1 | 118 |
| hHSF ms 6.10 | TATCAGATATTCTTCATCTTAGATTAGCGCAGTGGACTCAACCATTCCG | 1 | 119 |
| hHSF ms 6.16 | GCAGTCACGGAGACTCCTCGACGGCTCTCGTCGCCCACCC | 1 | 120 |
| hHSF ms 6.17 | TCTTGTAGACAGCTTCAATCTGCGTAATGTGAGGGATGTACGCAACT | 1 | 121 |
| hHSF ms 6.18 | CTAGACGGTAACGAGTGCCAATATAAAGTGGAATAGGGAATCCGCACGAA | 1 | 122 |
| hHSF ms 6.22 | AGGAGCACG | 1 | 123 |

### Discussion of Examples 1-9

In all SELEX approaches, the primary goal is to obtain aptamers that bind to a certain protein, usually a protein. Aptamers could be ligands to different protein domains, to the enzyme active site and substrate-binding centers, etc. However, because target biomolecules are labile to denaturation by heat or solvents, target stability is an important issue in SELEX experiment. Sol-gel technique has been proven to be applicable for target molecules immobilization in a biologically active form and provided for a high surface density for target compounds. Moreover, sol-gel processing has an enormous potential for applications such as immunological kits, drug delivery systems and biosensors (Fouque et al., Biosensors & Bioelectronics 22:2151-2157 (2007), which is hereby incorporated by reference in its entirety). One of the most important advantages of these sol-gels is the nano size pore formation. Two different types of pores have been observed on the sol-gel surface (data not shown). These pores, which are evenly distributed over the whole sol-gel surface work as molecular passages to immobilized proteins inside. That is, the nanoporous structure of the sol-gel matrix can allow for diffusion of some molecules such as aptamers, but it keeps target molecules, biomolecules immobilized in the pores.

Based on this, a strategy for selecting aptamers using sol-gel derived SELEX-on-a-chip device is described. Aptamer selection for TBP, which is component of Polymerase-II transcription machinery, was tested. TBP was immobilized with TFIIA, TFIIB, and hHSF1 as a competitor on SELEX-on-a-chip. The match between TBP aptamers selected by conventional and microfluidic SELEX demonstrated the effectiveness of using a microfluidic device to perform in vitro selection of aptamers against proteins or possibly against small molecule targets. In TBP aptamer selection, the microfluidic SELEX improves the efficiency of selection by reducing the number of cycle by 6. It takes 11 cycles to get the high affinity TBP aptamer pool with the conventional binding assay. Furthermore, aptamers can be selected even after the first cycle of microfluidic SELEX and without negative selection cycles. Modifications can be easily made and tested for larger protein immobilization by spot volume control, chamber space modification, unlimited circulation of aptamer library in the microfluidic chip using micropump, and connecting with other microscale separation service.

Currently, the SELEX process has been automated with the development of macrorobotic systems consisting of a PCR machine and a robotic manipulator to move reagents to multiple workstations (Cox et al., Bioorg Med Chem 9:2525-2531 (2001), Zhang et al., Nucleic Acids Symposium Series 219-220 (2000), which are hereby incorporated by reference in their entirety). In addition to platform development, an attractive feature of the SELEX devices is grafting of miniaturized platform. Hybarger et al. have reported the automated microline/valves based “start to finish” SELEX device (Hybarger et al., Anal Bioanal Chem 384:191-198 (2006), which is hereby incorporated by reference in its entirety). SELEX is still thought of as a method directed for a single target. Here, however, the multiplexed SELEX approach was introduced. Aptamers against TFIIA, TFIIB, and hHSF1 were sequenced and analyzed with TBP aptamer to compare the sequences among each aptamer set of four different proteins. There was one species that was common among the three sets for TFIIA, TFIIB, and hHSF I (SEQ ID NO: 84, see Tables 6-8). Some species seem to enrich from the microfluidic SELEX cycles. Theoretically, a large number of the proteins, depending upon the microfluidic system capacity, can be immobilized in this system. Furthermore, many proteins can work with each other as competitors for the selection of other aptamers. Through competition, only high affinity aptamers for the specific proteins can survive after the multiple cycles of in vitro selection.

### Example 10

### Design of Microfluidic Device Operable With 96-Chamber Format

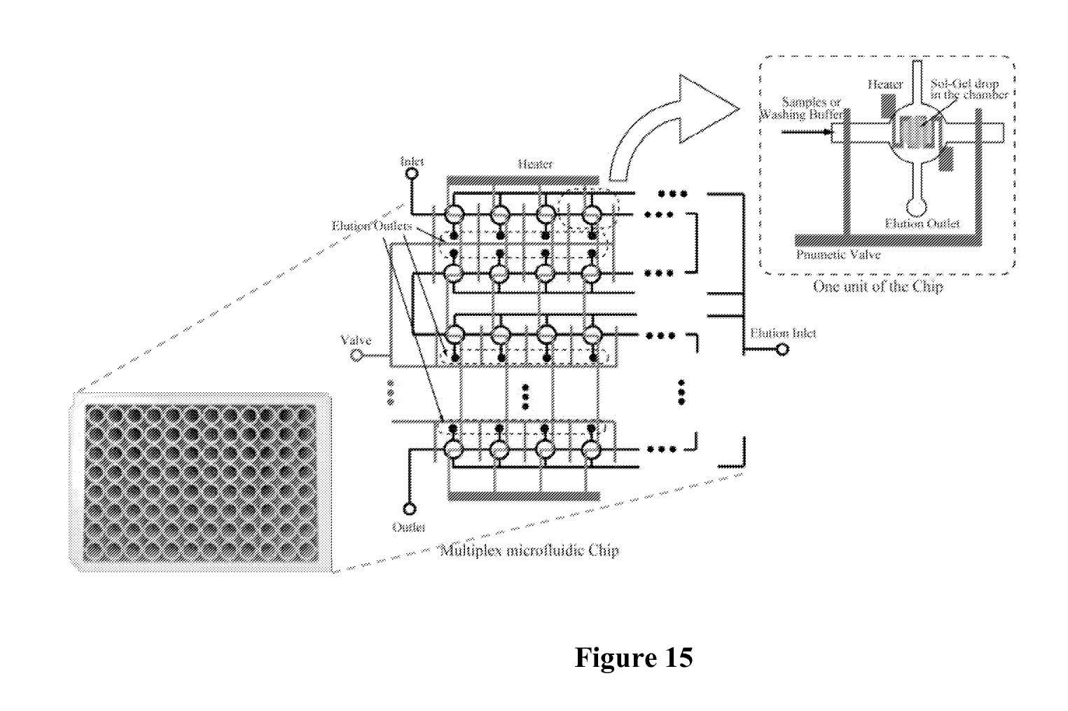

A 96-well format will allow for the construction of a microfluidic chip and system for performing multiplex SELEX against up to 96 distinct targets. The system design, illustrated in FIG. 15, shows that each chamber is adjacent to a microheater element and includes a pair of inlets and a pair of outlets for moving fluid into and out of each chamber. One inlet and one outlet are dedicated to elution and recovery of selected aptamers populations. Control over fluid movement is regulated by PDMS pump-valve system that includes one pneumatic valve controller and two pumps. This device will be constructed and used in separate experiments for screening a random aptamer population or an aptamer population previously selected by two rounds of conventional SELEX.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Additionally, the recited order of processing elements or sequences, or the use of numbers, letters, or other designations therefore, is not intended to limit the claimed processes to any order except as may be specified in the claims. The invention is also intended to cover any combinations of features, though separately described herein, unless their combination is explicitly excluded.

1. A microfluidic device comprising:
a substrate comprising one or more fluid channels extending between an inlet and an outlet,
a molecular binding region within the one or more fluid channels, wherein the molecular binding region comprises a target molecule; and
a heating element adjacent to the molecular binding region.

2. The microfluidic device according to claim 1, wherein the heating element comprises an electrode applied to a surface of the substrate.

3. The microfluidic device according to claim 1, wherein the substrate comprises one or more of glass, pyrex, glass ceramic, and polymer materials.

4. The microfluidic device according to claim 3, wherein the substrate is a combination of a glass or Pyrex base and a polymer lid, which together define the one or more fluid channels.

5. The microfluidic device according to claim 1, further comprising a polymer coating that encapsulates the heating element such that fluid passing through the fluid channels does not directly contact the heating element.

6. The microfluidic device according to claim 1, wherein the molecular binding region is formed on the polymer coating.

7. The microfluidic device according to claim 6, wherein the polymer coating is a poly(meth)acrylate.

8. The microfluidic device according to claim 1, wherein the molecular binding region comprises a high surface area material comprising the target molecule.

9. The microfluidic device according to claim 8, wherein the high surface area material is a sol-gel derived product, a hydrogel derived product, polymer brush derived product, nitrocellulose membrane encapsulation product, or dendrimer-based product.

10. The microfluidic device according to claim 1, wherein the molecular binding region comprises a surface of the one or more fluid channels comprising one or more linker molecules that tether the target molecule to the surface within said region.

11. The microfluidic device according to claim 1, wherein the target molecule is a protein or polypeptide, a carbohydrate, a lipid, a pharmaceutical agent, an organic non-pharmaceutical agent, or a macromolecular complex.

12. The microfluidic device according to claim 1 further comprising at least one chamber positioned between the inlet and outlet and in fluid communication with the one or more fluid channels, and a sol-gel material located substantially within the at least one chamber adjacent the heating element.

13. The microfluidic device according to claim 12, wherein the at least one chamber comprises two or more chambers.

14. The microfluidic device according to claim 13, wherein the two or more chambers comprise the same target molecule.

15. The microfluidic device according to claim 13, wherein the two or more chambers comprise different target molecules.

16. The microfluidic device according to claim 1 further comprising a multiport coupling in communication with the inlet.

17. The microfluidic device according to claim 16 further comprising one or more reservoirs in communication with the multiport coupling, the one or more reservoirs individually containing a wash buffer solution, a blocking buffer solution, a binding buffer solution, or a solution comprising a population of nucleic acid molecules.

18. A method of selecting a nucleic acid aptamer for binding to one or more target molecules comprising:
providing a microfluidic device according to claim 1
introducing a population of nucleic acid molecules into the microfluidic device under conditions effective to allow nucleic acid molecules to bind specifically to the target molecule;
removing from the microfluidic device substantially all nucleic acid molecules that do not bind specifically to the target molecule;
heating the heating element to cause denaturation of nucleic acid molecules that bind specifically to the target molecule; and
recovering nucleic acid molecules that bind specifically to the target molecule, the recovered nucleic acid molecules being aptamers that have been selected for their binding to the target molecule.

19. The method according to claim 18, wherein the nucleic acid aptamers comprise RNA aptamers, the method further comprising:
performing reverse transcription amplification of the selected aptamer population.

20. The method according to claim 19, further comprising:
purifying and sequencing the amplified aptamer population.

21. The method according to claim 20, wherein said recovering, said performing reverse transcription amplification, said purifying, and/or said sequencing are performed in one or more separate fluidic devices coupled in fluidic communication with the microfluidic device.

22. The method according to claim 18, wherein each of said introducing, removing, heating, and recovering is automated.

23. A nucleic acid aptamer identified in Tables 1-8, except that the aptamer is not one of SEQ ID NOS: 24, 70, and 81.

24. A method of selecting a nucleic acid aptamer for binding to one or more target molecules comprising:
providing a microfluidic device comprising:
a substrate comprising one or more fluid channels extending between an inlet and an outlet, and
one or more molecular binding regions within the one or more fluid channels, wherein the one or more molecular binding regions each comprises a target molecule;
introducing a population of nucleic acid molecules into the microfluidic device under conditions effective to allow the nucleic acid molecules to bind specifically to the one or more target molecules;
removing from the microfluidic device substantially all nucleic acid molecules that do not bind specifically to the target molecule(s);
denaturing the nucleic acid molecules that bind specifically to the target molecule(s); and
recovering nucleic acid molecules that bind specifically to the target molecule(s), the recovered nucleic acid molecules being aptamers that having been selected for their binding to the target molecule.

25. The method according to claim 24, wherein the one or more molecular binding regions comprise two or more molecular binding regions.

26. The method according to claim 25, wherein the two or more molecule binding regions are at discrete locations.

27. The method according to claim 26, wherein the two or more molecular binding regions comprise the same target molecule.

28. The method according to claim 26, wherein the two or more molecular binding regions comprise different target molecules.

29. The method according to claim 24, wherein the one or more regions contain a molecular complex comprising two or more target molecules.

30. The method according to claim 24, wherein said denaturing is carried out chemically.

31. The method according to claim 24, wherein said denaturing is carried out by locally heating the nucleic acid molecules bound specifically to the target molecules.

32. The method according to claim 24, wherein said denaturing and recovering is carried out separately for each of the one or more molecular binding regions.

33. The method according to claim 24, wherein the nucleic acid aptamers comprise RNA aptamers, the method further comprising:
performing reverse transcription amplification of the selected aptamer population.

34. The method according to claim 33, further comprising:
purifying and sequencing the amplified aptamer population.

35. The method according to claim 34, wherein said recovering, said performing reverse transcription amplification, said purifying, and/or said sequencing are performed in one or more separate fluidic devices coupled in fluidic communication with the microfluidic device.

36. The method according to claim 24, wherein each of said introducing, removing, denaturing, and recovering is automated.

37. A method of making a microfluidic SELEX device comprising:
applying a sol-gel material comprising a target molecule onto a surface of a first body component, and allowing solvent evaporation to occur, thereby forming a porous matrix comprising the target molecule; and
sealing a second body component onto the first body component, whereby the first and second body components together define a microfluidic device having an inlet, an outlet, and at least one microfluidic channel between the inlet and outlet, whereby the porous matrix is in fluid communication with the microfluidic channel.

38. The method according to claim 37 further comprising, prior to said applying the sol-gel material:
applying an electrode to the first body component and covering the electrode with a polymer, thereby forming the surface to which the sol-gel material is applied.

39. The method according to claim 38, wherein the electrode is a metal electrode.

40. The method according to claim 38, wherein said applying the electrode comprises:
applying a patterned photoresist layer on the first body component;
depositing metal onto the photoresist layer;
exposing the first body component to an electron beam evaporator to form a metal layer at regions of the first body component that lack the photoresist layer; and
removing the photoresist layer.

41. The method according to claim 38, wherein the polymer is a poly(meth)acrylate.

42. The method according to claim 37, wherein the first body component is formed of glass, pyrex, glass ceramic, or a polymer material and the second body component is formed of a polymer material.

43. The method according to claim 37, wherein the second body component comprises a relief pattern that forms the inlet, the outlet, and the at least one microfluidic channel upon said sealing.

44. A kit comprising the microfluidic device according to claim 1.

45. The kit according to claim 44, further comprising one or more of a random pool of nucleic acid molecules, wash buffer, binding buffer, blocking buffer, reagents for carrying out reverse transcription, PCR, and/or transcription, and directions for carrying out a SELEX process using the microfluidic device.
